(19) United States
(12) Patent Application Publication
(10) Pub. No.: US 2022/0193062 A1
(43) Pub. Date: Jun. 23, 2022

(54) COMBINATION OF ENZASTAURIN AND INHIBITORS OF BTK AND USES THEREOF

(71) Applicant: DENOVO BIOPHARMA LLC, San Diego, CA (US)

(72) Inventors: Yuqin Song, Beijing (CN); Yizi He, Beijing (CN); Yan Xie, Beijing (CN); Jun Zhu, Beijing (CN); Lingyan Ping, Beijing (CN); Wen Luo, San Diego, CA (US); Hong Sun, San Diego, CA (US)

(73) Assignee: DENOVO BIOPHARMA LLC, San Diego, CA (US)

(21) Appl. No.: 17/196,820

(22) PCT Filed: Sep. 6, 2019
(86) PCT No.: PCT/US2019/050104

§ 371 Date: Mar. 9, 2021

(30) Foreign Application Priority Data
Sep. 12, 2018 (CN) PCT/CN2018/105217

(51) Int. Cl. A61K 31/4545; A61K 31/519; A61K 31/4985; A61K 31/506; A61P 35/00

(52) U.S. Cl. CPC A61K 31/4545; A61K 31/519; A61P 35/00; A61K 31/506; A61K 31/4985

(57) ABSTRACT

The present invention relates to pharmaceuticals, particularly therapeutic combinations, pharmaceutical compositions and methods that comprise enzastaurin and a BTK inhibitor together. These combinations and methods for using these combinations provide therapeutic effects useful for treating various conditions including certain cancers, such as B-cell lymphatic cancers. Data provided herein demonstrates that enzastaurin and a BTK. inhibitor such as ibrutinib, when used together, can provide synergistic therapeutic effects.

### RELATED APPLICATION

The present application claims priority to PCT international patent application No. PCT/CN2018/105217, filed on Sep. 12, 2018, the disclosure of which is incorporated by reference in its entirety for all purposes.

### FIELD OF THE INVENTION

This invention relates to pharmaceutical compositions and combinations, and methods of using these compositions and combinations to treat conditions such as cancers, including lymphoma and related conditions, particularly B-cell lymphatic cancers. In particular embodiments, the invention provides combinations that comprise enzastaurin and an inhibitor of Bruton's tyrosine kinase (BTK), examples of which are disclosed herein, and methods of using these combinations to treat lymphoma.

### BACKGROUND OF THE INVENTION

Diffuse large B cell lymphoma (DLBCL), the most common form of lymphoma, is characterized by a heterogeneous tumor entity that can vary in morphologic, biological, immunophenotypic, and clinical presentation, and in therapeutic outcomes [1, 2]. According to gene expression profiling, two subtypes are common: germinal center B-cell like (GCB), and activated B-cell-like (ABC) subgroups of DLBCL. These two sub-types combined account for about 80% of DLBCL cases, leaving approximately 10-20% of cases “unclassified” [2]. ABC and GCB subtypes of DLBCL are involved in different cellular pathways, which poses a major barrier to understanding tumor development and maintenance, including its response to therapy [3]. Although durable remissions can be achieved in more than half of these patients, DLBCL remains a huge clinical challenge, with approximately 30% of patients not being cured [4]. Especially for relapsed/refractory DLBCL patients with poor survival rates, novel and effective therapeutic strategies are urgently needed.

Abnormal B-cell receptor (BCR) signaling has been implicated in the pathogenesis of B-cell malignancy, which is widely appreciated as one of the main mechanisms that promote disease progression [5, 6]. Continuous activation of BCR in DLBCL leads to the phosphorylation and activation of regulatory and adaptor proteins, such as spleen tyrosine kinase (SYK), Bruton's tyrosine kinase (BTK), and protein kinase C-β (PKCβ), especially in ABC-type DLBCL [3, 7, 8]. By contrast, oncogenic signaling in GCB DLBCL is initiated and reinforced by sharing a dependence on PI3K/mTOR signaling, which is independent of nuclear factor κB (NF-κB) [9, 10]. In recent years, an increasing amount of research has focused on the therapeutic inhibition of BCR signaling, especially combination-based therapeutic regimens for treating DLBCL [7, 11, 12].

DLBCL is a heterogeneous lymphoma, and while the introduction of rituximab has greatly improved the outcome for many, about 30%˜40% of all cases remain incurable [32]. One important reason for this situation is that ABC and GCB DLBCLs involve different signaling pathways, as mentioned above. A prominent feature of the ABC subtype is harboring mutations in MYD88, CARD11, CD79A and CD79B, which are characterized by a constitutively promoted NF-κB pathway signaling, exhibiting less favorable clinical outcomes [8, 33, 34]. In contrast, GCB subtype is more dependent on PI3K/AKT activity rather than the NF-κB pathway [10]. This signal diversity translates into different levels of tumor aggressiveness and differing response to therapeutic approaches [35]. Thus BCR inhibiting agents, including inhibitors of BTK, PI3K, SYK, and PKCβ, represent a promising therapeutic strategy for DLBCL patients. Data herein demonstrates for the first time that combination treatment with enzastaurin and ibrutinib (a BTK inhibitor) leads to augmented anti-tumor effects on DLBCL both in vitro and in vivo. Mechanistic data suggest that this effect may rely on inactivation of related signaling pathways and down-regulating NOTCH1 expression.

Enzastaurin is a relatively well-studied anti-tumor agent. It targets PKCβ with an IC50 of 6 nmol/L, and also inhibits other PKC isoforms at higher concentrations. Preclinical research on enzastaurin has produced promising results in cutaneous T-cell lymphoma, B-cell lymphoma, multiple myeloma (MM), Waldenstrom's macroglobulinemia (WM), and other solid tumors [36-39]. Previous research has established that 22% of DLBCL tumor samples are positive for PKCβ expression as defined by immunostaining of >50% of cells; furthermore, PKCβ expression is a useful marker of poor prognosis in DLBCL [40, 41]. Phase I/II studies showed that enzastaurin is well tolerated in patients and 15% (8/55) of the patients had prolonged freedom from progression (FFP≥4 cycles) and 7% (4/55) of the patients even experience FFP 20˜50 months [18, 19]. However, in a phase III clinical trial (PRELUDE), enzastaurin alone did not significantly improve disease-free survival (DFS) in high-risk DLBCL patients after remission of B cell lymphoma. This halted the development of enzastaurin as monotherapy in DLBCL.

Analysis of failed therapeutics presents an opportunity to advance both preclinical and clinical investigations, because it may provide insights toward the development of therapeutic combinations. Prior studied have noted the combination treatment with HDAC inhibitors (HDACi) and enzastaurin exhibit a synergistic effect in DLBCL, for example, as HDACi may increase the expression of PKCβ leading to an activation of survival signals [16]. Additionally, therapeutic regimens comprising enzastaurin combined with other agents like lenalidomide, NVP-BEZ235 (PI3K inhibitor), and bortezomib have been exploited to treat non-Hodgkin lymphoma cell lines [14, 42, 43]. These are examples of attempts to identify therapeutic combinations to utilize agents that did not exhibit single-agent efficacy sufficient for development despite having potentially valuable in vitro activity.

There remains a need for new treatments for B-cell lymphatic cancers such as DLBCL, particularly combinations that target multiple biochemical pathways and thus are better able to treat heterogeneous tumors and combat resistance mechanisms. The invention provides such combinations and methods for their use.

### SUMMARY OF THE INVENTION

Enzastaurin, a potent and selective orally administered inhibitor of several PKC isoforms, was shown to regulate the PI3K/AKT/mTOR, MAPK, and JAK/STAT pathways in solid and hematological malignancies [13-16]. Interestingly, some researchers have found that PKCβ works as a feedback loop inhibitor of BTK activation, which modulates signaling pathway via altering BTK membrane localization [23, 24]. PKCβ can down-regulate BTK's activation via both transphosphorylation at Tyr551 and autophosphorylation at Tyr223. Besides, inhibition of PKCβ leads to enhanced membrane targeting of BTK, up-regulated phosphorylation of PLCγ2, and amplified BCR-mediated Ca²+ signaling [24].

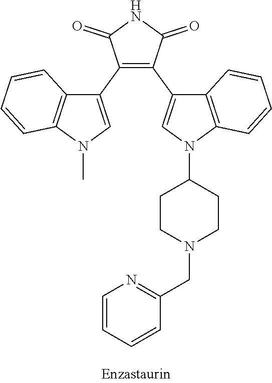 [SMILES: Cn1cc(C2=C(c3cn(C4CCN(Cc5ccccn5)CC4)c4ccccc34)C(=O)NC2=O)c2ccccc21]

Although the effect of enzastaurin in DLBCL was confirmed in preclinical studies and Phase I/II clinical trials of DLBCL, its Phase III clinical trials did not meet the primary end point [17-19], leading the sponsor to discontinue clinical trials. While several attempts have been made to show therapeutic effects with enzastaurin in different clinical trials, it has not been approved for any therapeutic uses in the U.S.

Ibrutinib (PCI-32765) is an orally active inhibitor of BTK inhibitor that binds Cysteine-481 on the kinase domain of BTK, leading to an irreversible inhibition at Tyr-223. Significant progress has been made in the development of ibrutinib in recent years, demonstrating efficacy in a variety of B-cell malignancies. In ABC and GCB sub-types of DLBCL, signal pathway differences translate to differences in response to BTK, which is largely confirmed in a Phase II trial of ibrutinib in relapsed DLBCL patients. The results revealed that 37% of patients (14/38) with ABC-DLBCL but only 5% (1/20) of patients with GCB -DLBCL produced overall response rate (ORR) [3]. Beyond that, the ABC-DLBCL patients harboring CD79A/Bmut, CARD11mut, TNFAIP3mut, or MYD88mut showed primary resistance to ibrutinib [3, 7]. A subset of patients that initially responded well after treatment with ibrutinib eventually relapsed, apparently due to activating mutations in BTK or PLC-γ2 (the protein immediately downstream of BTK). This underscores the need for developing new target agents and combination treatments to improve the outcomes of treatments when resistance is present due to tumor heterogeneity or develops as a result of treatment [44]. More recent attention has been focused on drug combinations, particularly co-treatment with BTK inhibitors and lenalidomide, bortezomib, PI3K inhibitors, and Pan-SRC kinase inhibitors in DLBCL [7, 12, 45- 48]. When ibrutinib was added to DLBCL cells treated with these agents, the drug exhibited synergistic cytotoxic effects on cells. There is also clinical data supporting ibrutinib use in combination therapy with rituximab and ofatumumab for the treatment of relapsed or refractory CLL/SLL [49]. Current on-going trials will further define the role of ibrutinib as upfront therapy and/or as a combination treatment in B-cell lymphoid malignancies.

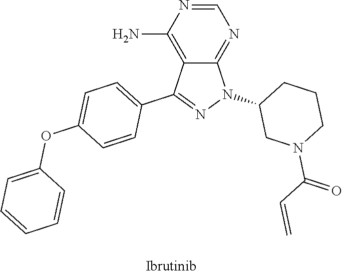 [SMILES: C=CC(=O)N1CCC[C@@H](n2nc(-c3ccc(Oc4ccccc4)cc3)c3c(N)ncnc32)C1]

Data herein demonstrates that the combination of the PKCβ inhibitor enzastaurin and a BTK inhibitor such as ibrutinib produces synergistic anti-tumor effects in DLBCL. The combination of enzastaurin and low doses of ibrutinib (e.g., lower than single-agent dose) act synergistically to suppress DLBCL cell growth in vitro and in vivo. Based on the results, the combination of enzastaurin with an inhibitor of BTK such as ibrutinib, would be an effective therapeutic treatment for patients with DLBCL, independent of molecular subtypes and signaling dependencies, and is expected to be effective for treatment of other malignancies of the cellular immune system, especially for malignancies of B-cell origin.

Moreover, based on data provided herein, enzastaurin appears able to enhance the efficacy of a BTK inhibitor for therapeutic uses generally. Without being bound by theory, it is believed that the biochemical interaction allows enzastaurin to exhibit synergy when used in combination with a BTK inhibitor for treating oncologic conditions, immunological disorders, gastrointestinal disorders, CNS disorders, dermatological disorders, hematological disorders, and metabolic disorders.

Immunological disorders treatable with the combinations of the invention include graft versus host disease (GVHD), rheumatoid arthritis, systemic lupus erythematosus, pemphigus vulgaris, Sjogren's Syndrome, and other autoimmune disorders.

Gastrointestinal disorders treatable with the combinations of the invention include systemic mastocytosis.

CNS disorders treatable with the combinations of the invention include multiple sclerosis, particularly relapsing multiple sclerosis.

Dermatological disorders treatable with the combinations of the invention include chronic urticaria.

Hematological disorders treatable with the combinations of the invention include thrombocytopenic purpura.

Oncology indications treatable with the combinations of the invention include chronic lymphocytic leukemia (CLL), extranodal marginal zone B -cell lymphoma, mucosa-associated lymphoid tissue lymphoma (MALT-lymphoma), Waldenstrom's macroglobulinemia, mantle cell lymphoma, relapsed CLL, refractory CLL, follicular lymphoma, adenocarcinoma, metastatic adenocarcinoma (e.g., pancreatic), non-Hodgkin lymphoma, pancreatic cancer, acute lymphocytic leukemia, acute lymphoblastic leukemia, hairy cell leukemia, metastatic breast cancer, acute myelocytic leukemia, acute myeloblastic leukemia, multiple meloma, refractory multiple myeloma, relapsed multiple myeloma, gastric cancer, colorectal cancer, bladder cancer, Hodgkin lymphoma (B-cell Hodgkin lymphoma), metastic melanoma, non-small cell lung cancer, primary CNS lymphoma, renal cell carcinoma, secondary CNS lymphoma, transitional cell carcinoma, urothelial cell carcinoma, nodal marginal B-cell lymphoma, splenic marginal zone B-cell lymphoma, T-cell lymphomas, epithelial ovarian cancer, fallopian tube cancer, peritoneal cancer, (recurrent) head and neck cancer, squamous cell carcinoma, (recurrent) glioblastoma multiforme (GBM), and B-Cell lymphoma including diffuse large B-cell lymphoma.

In one aspect, the invention provides a method to treat a disease or condition selected from oncologic conditions, immunological disorders, gastrointestinal disorders, CNS disorders, dermatological disorders, hematological disorders, and metabolic disorders. The method comprises administering to a subject in need of such treatment, an effective amount of enzastaurin and a BTK inhibitor; preferably the method comprises administering enzastaurin and a BTK inhibitor in amounts sufficient to provide synergistic effectiveness. In some embodiments, the invention provides a method to treat cancers, particularly B -cell related cancers.

In one aspect, the disclosure provides a method to treat lymphoma and related conditions, which comprises administering to a subject in need thereof enzastaurin or a pharmaceutically acceptable salt thereof, and a second therapeutic agent, where the second therapeutic agent is an inhibitor of Bruton's tyrosine kinase (BTK). In some embodiments, the methods are used to treat lymphoma, particularly DLBCL.

In another aspect, the disclosure provides a composition that comprises enzastaurin or a pharmaceutically acceptable salt thereof and a BTK inhibitor, which is typically a low molecular weight organic compound, e.g., one having molecular weight between 200 and about 2000. Optionally, the compositions can include a pharmaceutically acceptable carrier or excipient. Suitably, the BTK inhibitor can be ibrutinib.

In another aspect, the disclosure provides a therapeutic combination comprising enzastaurin or a pharmaceutically acceptable salt thereof, and a BTK inhibitor. The two therapeutic agents (enzastaurin and the BTK inhibitor) can be administered together or separately; commonly, they are in separate dosage units (pills or capsules, for example), which may be taken together or at different times. Each component can be separately prepared for administration, or the two can be combined into a single composition.

The BTK inhibitor for the foregoing aspects can be selected from M7583, ibrutinib, acalabrutinib, zanubrutinib, CT-1530, DTRMWXHS-12, spebrutinib besylate, vecabrutinib, evobrutinib, tirabrutinib, fenebrutinib, poseltinib, BMS-986142, ARQ-531,LOU-064, PRN-1008, ABBV-599, AC-058, ARQ-531, BIIB-068, BMS-986195, HWH-486, PRN-2246, TAK-020, GDC-0834, BMX-IN-1, RN486, SNS-062, LFM-A13, PCI-32765 (racemate of ibrutinib), CGI-1746, ONO-4059, and SHR-1459, or a pharmaceutically acceptable salt of one of these. The compositions, combinations and methods of the invention can be practiced with any of these BTK inhibitors or with a mixture of two or more of them, or with pharmaceutically acceptable salts of these BTK inhibitors.

GDC-0834 is a potent and selective BTK inhibitor with an IC50 of 5.9 and 6.4 nM in in vitro enzyme and cell experiments, respectively, and exhibited in vivo IC50 of 1.1 and 5.6 μM in mice and rats, respectively. BMX-IN-1 is a selective, irreversible bone marrow tyrosine kinase on chromosome X (BMX) inhibitor. In the BMX ATP binding domain, the compound targets Cys496 with an IC50 value of 8 nM; and in BTK, its IC50 value is 10.4 nM. RN486 is a highly active Btk inhibitor with an IC50 of 4.0 nM. SNS-062 is a potent, non-covalent inhibitor of BTK and interleukin-2-inducible T-cell kinase (ITK) inhibitor with Kd values of 0.3 nM and 2.2 nM, respectively; SNS-062 has an IC50 of 24 nM for ITK. LFM-A13 is a potent BTK, JAK2, PLK inhibitor, that inhibits the activity of BTK, Plx1 and PLK3 with IC50 of 2.5 μM, 10 μM and 61 μM, respectively. PCI-32765 is a racemic form of ibrutinib, and is a selective inhibitor of Btk with an IC50 of 0.5 nM; it exhibits moderate inhibition of Bmx, CSK, FGR, BRK, and HCK, and lower activity on EGFR, Yes, ErbB2, and JAK3. CGI-1746 is a potent, highly selective BTK inhibitor with an IC50 of 1.9 nM. ONO-4059 has an IC50 value of 2.2 nm and is a selective BTK inhibitor. In B cells, ONO-4058 binds to BTK, thus blocking B cell receptor signaling and impeding the development of B cells. QL47 is an irreversible BTK inhibitor with an IC50 of 7 nM.

Preferably the inhibitor of BTK is selected from M7583, Ibrutinib, and Acalabrutinib, or a pharmaceutically acceptable salt thereof. Ibrutinib is a preferred BTK inhibitor for the compositions, combinations and methods.

In another aspect, the invention provides an in vivo therapeutic combination, which is a mixture comprising enzastaurin and a BTK inhibitor that forms in vivo in a subject when enzastaurin, or a pharmaceutically acceptable salt thereof, and a BTK inhibitor such as ibrutinib, or a pharmaceutically acceptable salt thereof, are administered to the subject contemporaneously. Administration of the two actives is contemporaneous when they are administered together, or when both are administered within a period of one hour, or within a period of two hours, or when they are administered closely enough together in time for both to be present simultaneously in the plasma or blood of the subject at a level of at least 5% and typically at least 10% of the Cmax for each of the individual components. Preferably, the therapeutic combination comprises simultaneous blood or plasma concentrations of at least about 20% of the Cmax for each of the components (enzastaurin and the BTK inhibitor used). The Cmax in this context refers to the maximum blood or plasma concentration seen when the component is administered alone, using the same route of administration, dosing and formulation used for that component in the combination therapy.

As used herein, ‘BTK inhibitor’ and ‘inhibitor of BTK’ are intended to have the same meaning, and unless explicitly otherwise indicated, the terms include pharmaceutically acceptable salts.

The compositions and methods herein can be used to treat any suitable condition, most typically for the treatment of B-cell lymphatic disorders such as Hodgkin's and non-Hodgkin's lymphoma and mantle cell lymphoma. Data herein shows the methods and compositions are particularly useful to treat diffuse large B-cell lymphoma (DLBCL).

In some embodiments, the subject to be treated is one having been diagnosed with a B-cell proliferative disorder, such as a form of lymphoma. In some embodiments, the subject is selected based on the presence of a biomarker, such as DGM1 (Denovo Genetic Marker 1).

In some embodiments, the methods and compositions are used in combination with at least one additional therapeutic agent useful for treating the subject to be treated with the combinations of the invention. As an example, the subject may be treated with a conventional chemotherapeutic agent useful to treat the same condition in the subject, such as rituximab, or the subject may be treated with the combinations of the invention in conjunction with a combination treatment such as CHOP, a conventional chemotherapy regimen that includes the drugs cyclophosphamide, doxorubicin hydrochloride (hydroxydaunorubicin), vincristine sulfate (Oncovin), and prednisone. In other embodiments, the methods and compositions comprising enzastaurin and a BTK inhibitor can be used along with R-CHOP, which is an abbreviation for a chemotherapy combination that is used to treat non-Hodgkin lymphoma and mantle cell lymphoma and is being studied in the treatment of other types of cancer. R-CHOP includes the drugs rituximab, cyclophosphamide, doxorubicin hydrochloride (hydroxydaunorubicin), vincristine sulfate (Oncovin), and prednisone. Other therapeutic agents that may be used with the combination of enzastaurin and a BTK inhibitor include, for example, lenalidomide, bortezomib, and PI3K inhibitors such as BEZ235.

Also, as is increasingly common, the therapeutic combinations disclosed herein can be administered in conjunction with an immunooncology therapeutic agent, such as a PD-1 or PD-L1 inhibitor, or other known checkpoint inhibitors, that help the body's own immune system recognize and combat cancer cells. The checkpoint inhibitors assist the subject's immune system in recognizing and attacking abnormal cells, such as cancerous cells, and can significantly boost the efficacy of chemotherapies such as the combinations of enzastaurin and a BTK inhibitor as disclosed herein. Suitable checkpoint inhibitors include biologics as well as small-molecule therapeutics; examples of these include ipilimumab, nivolumab, atezolizumab, avelumab, pembrolizumab, tislelizumab, and durvalumab.

Any suitable BTK inhibitor can be used in combination with enzastaurin for the compositions, combinations and methods of the invention. The BTK inhibitor for the foregoing aspects can be selected from M7583, ibrutinib, acalabrutinib, zanubrutinib, CT-1530, DTRMWXHS-12, spebrutinib besylate, vecabrutinib, evobrutinib, tirabrutinib, fenebrutinib, poseltinib, BMS-986142, ARQ-531,LOU-064, PRN-1008, ABBV-599, AC-058, ARQ-531, BIIB -068, BMS-986195, HWH-486, PRN-2246, TAK-020, GDC-0834, BMX-IN-1, RN486, SNS-062, LFM-A13, PCI-32765 (racemate of ibrutinib), CGI-1746, ONO-4059, and SHR-1459, or a pharmaceutically acceptable salt of one of these. Preferably the inhibitor of BTK is selected from M7583, Ibrutinib, and Acalabrutinib, or a pharmaceutically acceptable salt thereof. Ibrutinib is a preferred BTK inhibitor for the combinations, compositions and methods of the invention.

The compositions and methods are preferably used for treating DLBCL.

In data herein, the combination of PKCβ inhibitor enzastaurin and BTK inhibitor ibrutinib shows synergistic anti-tumor effects in ABC and GCB types of DLBCL, thereby providing rationale for preclinical and clinical investigations of these combinations, and allowing for the development of specific, well tolerated and efficient cancer therapeutics for DLBCL, and especially for subjects having relapsed or refractory DLBCL.

Some studies have demonstrated the role of PKCβ in the negative regulation of BTK, and PKCβ inhibitors alter phosphorylation levels of BTK, which leads to enhanced BTK signaling [23, 24]. The results disclosed herein support that association. The expression of p-BTK exhibited a marked increase following treatment with enzastaurin. Thus, PKCβ potently acts as negative feedback signal of BTK, which means a PKCβ inhibitor can upregulate BTK's activation and change oncogenic signals downstream of the BCR. This may explain why the combination of the PKCβ inhibitor enzastaurin and the BTK inhibitor ibrutinib has synergistic anti-tumor effects in DLBCL. The synergistic anti-tumor effects of these two agents are seen at concentrations lower than their IC50 values, and include reduction of proliferation, promoting apoptosis, inducing G1 phase arrest, preventing cellular invasion and migration, and down-regulating activation of downstream signaling. Without being bound by theory, it is believed this relationship causes enzastaurin to increase the therapeutic potency of BTK inhibitors, producing synergy as described herein.

The downstream signaling cascades study also demonstrated that the combination treatment with enzastaurin and a BTK inhibitor such as ibrutinib triggers a time-dependent inhibition of mRNA levels of NOTCH1, whereas either of these drugs alone just slightly affects the expression of NOTCH1. NOTCH1 belongs to a family of transmembrane receptors that directly transduces extracellular signals into gene expression changes [29]. The oncogenic capacity of NOTCH1 has been verified in hematological diseases, including T-cell acute lymphoblastic leukemia, multiple myeloma (MM), Hodgkin and anaplastic large cell lymphoma [27, 28, 30]. Many recent studies also have shown that a large number of DLBCL patients harbor NOTCH1 mutations and aberrations, which emphasizes the oncogenic role of NOTCH1 as the genetic drivers of DLBCL [50-52]. Moreover, NOTCH1 facilitates the activation of the PI3K-AKT-mTOR and NF-KB signaling pathways, which play an important role in accelerating growth and inhibiting apoptosis, not only in T-cell neoplasms, but also in B-cell neoplasms [28, 29]. The present studies show that treatment of DLBCL with a combination of enzastaurin and ibrutinib significantly reduced gene expression of NOTCH1. The shRNA mediated reduction in NOTCH1 expression dramatically inhibited DLBCL cell proliferation. These data indicate that down-regulating of NOTCH1 could be a crucial biological mechanism underlying the synergistic effect of co-treatment with enzastaurin and a BTK inhibitor in suppressing cell growth. These studies may explain why enzastaurin exhibits synergistic activity when used in combination with a BTK inhibitor.

Regardless, without being bound by theories regarding the mechanism, the synergistic effectiveness of the combination of enzastaurin and ibrutinib in DLBCL in vitro and in vivo demonstrated herein shows that co-treatment with enzastaurin and an inhibitor of BTK produces anti-tumor effects in DLBCL, independent of molecular subtype. These results demonstrate that such combinations are a viable therapeutic treatment, and the simultaneous suppression of BTK and PKCβ appears to be a new approach for treating both ABC and GCB sub-types of DLBCL.

The compositions and methods described herein can be used for any suitable purpose. In some embodiments, the compositions described above can be used in therapy, particularly for treatment of B-cell related malignancies such as lymphoma. As shown, the synergistic effect of the combination applies to both ABC and GCB forms of DLBCL.

In still another aspect, the present disclosure provides pharmaceutical compositions comprising enzastaurin and a BTK inhibitor such as those as described herein. In these embodiments, enzastaurin and the BTK inhibitor are often admixed with at least one pharmaceutically acceptable carrier or excipient. In some embodiments, enzastaurin and the BTK inhibitor are admixed with at least two pharmaceutically acceptable carriers or excipients.

In yet another aspect, the present disclosure provides a method for treating and/or preventing a B-cell lymphatic disorder such as lymphoma, which comprises administering to a subject in need thereof an effective amount of a combination as described above, comprising enzastaurin and a BTK inhibitor such as ibrutinib, or a pharmaceutical composition containing these substances as described herein. Enzastaurin and the BTK inhibitor can optionally be used in the form of pharmaceutically acceptable salts. In some embodiments the B-cell lymphatic disorder is DLBCL, including ABC and GCB subtypes of DLBCL. In some embodiments of these methods, the subject is selected based on a biomarker, such as the presence of biomarker DGM1.

In yet another aspect, the present disclosure provides a method for reducing the risk of metastasis or relapse in a subject having been treated for a B-cell lymphatic disorder such as lymphoma, which comprises administering to a subject in need thereof an effective amount of a combination as described above, comprising enzastaurin and a BTK inhibitor such as ibrutinib, or a pharmaceutical composition containing these substances as described herein. Enzastaurin and the BTK inhibitor can optionally be used in the form of pharmaceutically acceptable salts. In some embodiments the B-cell lymphatic disorder is DLBCL, including ABC and GCB subtypes of DLBCL. In some embodiments of these methods, the subject is selected based on a biomarker, such as the presence of biomarker DGM1.

In another aspect, the invention provides a therapeutic combination for use in therapy, in particular for therapeutic treatment of lymphoma such as DLBCL, where the combination comprises enzastaurin and a BTK inhibitor selected from those disclosed herein. The therapeutic combination can be a single pharmaceutical composition containing both enzastaurin and the BTK inhibitor, or the combination can be two separate pharmaceutical compositions for use together but able to be administered separately. The therapeutic combination can also be produced in vivo, upon the administration of enzastaurin and a BTK inhibitor such as ibrutinib to a subject in a manner to cause both enzastaurin and the BTK inhibitor to be simultaneously present at relevant plasma or blood concentrations.

In yet another aspect, the present disclosure provides for a use of a therapeutic combination described above, e.g., enzastaurin and ibrutinib, for the manufacture of a medicament. While it is understood that the two active therapeutic agents can be administered separately, in some embodiments of the invention, they are formulated together into a single dosage unit for administration as a medicament, especially a medicament for treating lymphoma, including DLBCL.

In yet another aspect, the present disclosure provides a combination of enzastaurin and a BTK inhibitor such as ibrutinib, for use to treat and/or prevent a lymphocytic cancer, preferably, a B-cell lymphoma such as DLBCL.

In yet another aspect, the present disclosure provides a combination of enzastaurin and a BTK inhibitor such as ibrutinib, for use to reduce the risk of metastasis or relapse in a subject having been treated for a lymphocytic cancer, particularly a B-cell lymphoma such as DLBCL.

In yet another aspect, the present disclosure provides a method for treating and/or preventing a lymphocytic cancer, preferably, a B-cell lymphoma such as DLBCL, which methods comprises administering to a subject in need thereof an effective amount of the combination described above. In some embodiments, the subject is selected based on the level of expression or presence of a biomarker such as DGM1 (Denovo Genetic Marker 1). DGM1 and its use as a biomarker for selecting subjects for treatment with enzastaurin are disclosed and described in published patent application WO2018/045240, and the methods can be used similarly for selecting subjects to be treated with the therapeutic combinations herein, e.g. enzastaurin and ibrutinib.

In yet another aspect, the present disclosure provides a method for inhibiting an activity of a Bruton's tyrosine kinase (Btk or BTK) and PKCβ, and the respective pathways, in a cell, organ or tissue, which methods comprises contacting BTK or a cell, organ or tissue, with an effective amount of a combination of enzastaurin and a BTK inhibitor, e.g., ibrutinib, as described above, or a pharmaceutical composition comprising the combination as described above.

In yet another aspect, the present disclosure provides an use of a combination of enzastaurin, or a pharmaceutically acceptable salt thereof, and an inhibitor of BTK for the manufacture of a medicament for treating or preventing a disorder or disease selected from oncologic conditions, immunological disorders, gastrointestinal disorders, CNS disorders, dermatological disorders, hematological disorders and metabolic disorders in a subject in need of such treatment or prevention.

In yet another aspect, the present disclosure provides an use of a combination of enzastaurin, or a pharmaceutically acceptable salt thereof, and an inhibitor of BTK for the manufacture of a medicament for treating or preventing lymphoma in a subject in need of such treatment or prevention, or for reducing risk of metastasis or relapse in a subject having been treated for lymphoma.

### BRIEF DESCRIPTION OF THE DRAWINGS

FIG. 1 (including FIGS. 1a-1c) shows inhibition of ABC and GCB cell lines by enzastaurin, and upregulation of BTK phosphorylation.

FIG. 2 (including FIGS. 2a-2c) shows synergy when enzastaurin and ibrutinib are used together in DLBCL cells.

FIG. 3 (including FIGS. 3a-3d) shows that the combination of enzastaurin and ibrutinib promoted apoptosis and induced G1 phase arrest in DLBCL cells.

FIG. 4 (including FIGS. 4a-4d) shows the combination of enzastaurin and ibrutinib synergistically inhibits migration and invasion by DLBCL cells.

FIG. 5 shows synergy in the inhibition of downstream signaling by enzastaurin and ibrutinib in three cell lines.

FIG. 6 (including FIGS. 6a-6f) shows whole-transcriptosome changes in DLBCL caused by the combination of enzastaurin and ibrutinib.

FIG. 7 (including FIGS. 7a-7d) shows synergistic antitumor effects with enzastaurin and ibrutinib in DLBCL-derived xenograft tumors.

FIG. 8 (including FIGS. 8a-8c) shows SU-DHL-6 cell growth inhibition by Enzastaurin and BTK inhibitors alone or in combination at the concentrations of Enzastaurin at 1 μM, 3 μM and 5 μM for 72 hours. Three BTK inhibitors, Zanubrutinib, Acalabrutinib, and ARQ531 were tested in alone or combination assays. Data are expressed as compound inhibition effects of cells treated with vehicle control. Results represent the Mean±SEM for the triplicates of each treatment. *P<0.05, **P<0.01.

FIG. 9 (including FIGS. 9a-9b) shows synergistic effects of Enzastaurin and BTK inhibitor Vecabrutinib in SU-DHL-5 (top graph) and SU-DHL-6 (bottom graph) cell growth inhibitions assays. The constant ratio concentration method was used for combined drug dose selection. The cells were treated with Enzastaurin (0.08-5 μM) and Vecabrutinib (0.06-4 μM) alone or in combination of same ratio for 72 h in triplicates of each treatment. The combination Index (CI) values were calculated and listed in above graphs for evaluating Enzastaurin synergistic effects. The results demonstrated synergistic activities of 2 drugs with CI<1 in all tested doses except Enzastaurin at 2.5 μM in SU-DHL-6 cells.

### DETAILED DESCRIPTION

### General Definitions

Unless defined otherwise, all technical and scientific terms used herein have the same meaning as is commonly understood by one of ordinary skill in the art to which this invention belongs. All patents, applications, published applications and other publications referred to herein are incorporated by reference in their entireties. If a definition set forth in this section is contrary to or otherwise inconsistent with a definition set forth in a patent, application, or other publication that is herein incorporated by reference, the definition set forth in this section prevails over the definition incorporated herein by reference.

As used herein, “a” or “an” means “at least one” or “one or more”.

“Treating” or “treatment” or “alleviation” refers to therapeutic treatment wherein the object is to slow down (lessen) if not cure the targeted pathologic condition or disorder or prevent recurrence of the condition. A subject is successfully “treated” if, after receiving a therapeutic amount of a therapeutic agent or treatment, the subject shows observable and/or measurable reduction in or absence of one or more signs and symptoms of the particular disease. Reduction of the signs or symptoms of a disease may also be felt by the patient. A patient is also considered treated if the patient experiences stable disease. In some embodiments, treatment with a therapeutic agent is effective to result in the patients being disease-free 3 months after treatment, preferably 6 months, more preferably one year, even more preferably 2 or more years post treatment. In some embodiments, treatment with a therapeutic agent is effective to result in longer survival time and/or better survival rate for the patients, e.g., increasing the Overall Survival of the patients. These parameters for assessing successful treatment and improvement in the disease are readily measurable by routine procedures familiar to a physician of appropriate skill in the art. In some embodiments, “treatment” means any manner in which the symptoms of a condition, disorder or disease are ameliorated or otherwise beneficially altered. Treatment also encompasses any pharmaceutical use of the compositions herein. In some embodiments, “amelioration” of the symptoms of a particular disorder by administration of a particular pharmaceutical composition refers to any lessening, whether permanent or temporary, lasting or transient that can be attributed to or associated with administration of the composition.

The term “prediction” or “prognosis” is often used herein to refer to the likelihood that a patient will respond either favorably or unfavorably to a drug or set of drugs, or the likely outcome of a disease. In one embodiment, the prediction relates to the extent of those responses or outcomes. In one embodiment, the prediction relates to whether and/or the probability that a patient will survive or improve following treatment, for example treatment with a particular therapeutic agent, and for a certain period of time without disease recurrence. The predictive methods of the invention can be used clinically to make treatment decisions by choosing the most appropriate treatment modalities for any particular patient. The predictive methods of the present invention are valuable tools in predicting if a patient is likely to respond favorably to a treatment regimen, such as a given therapeutic regimen, including for example, administration of a given therapeutic agent or combination, surgical intervention, steroid treatment, etc.

As used herein the language “pharmaceutically acceptable carrier” is intended to include any and all solvents, dispersion media, coatings, isotonic and absorption delaying agents, and the like, compatible with pharmaceutical administration. The use of such media and agents for pharmaceutically active substances is well known in the art. See, e.g., Remington, The Science and Practice of Pharmacy, 20^{th} ed., (Lippincott, Williams & Wilkins 2003). Except insofar as any conventional media or agent is incompatible with the active compound, such use in the compositions is contemplated.

A “pharmaceutically acceptable salt” is intended to mean a salt of a free acid or base of a compound represented herein that is non-toxic, biologically tolerable, or otherwise biologically suitable for administration to a subject. See, generally, Berge, et al., J. Pharm. Sci., 1977, 66, 1-19. Preferred pharmaceutically acceptable salts are those that are pharmacologically effective and suitable for contact with the tissues of subjects without undue toxicity, irritation, or allergic response. Enzastaurin and inhibitors of Bruton's tyrosine kinase (BTK) described herein may possess a sufficiently acidic group, a sufficiently basic group, both types of functional groups, or more than one of each type, and accordingly react with a number of inorganic or organic bases, and inorganic and organic acids, to form a pharmaceutically acceptable salt.

In some embodiments, the term “pharmaceutically acceptable salt” means a salt which is acceptable for administration to a patient, such as a mammal, such as human (salts with counterions having acceptable mammalian safety for a given dosage regime). Such salts can be derived from pharmaceutically acceptable inorganic or organic bases and from pharmaceutically acceptable inorganic or organic acids. In some embodiments, “pharmaceutically acceptable salt” refers to pharmaceutically acceptable salts of a compound, which salts are derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, formate, tartrate, besylate, mesylate, acetate, maleate, oxalate, and the like.

Examples of pharmaceutically acceptable salts include sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, methylsulfonates, propylsulfonates, besylates, xylenesulfonates, naphthalene-1-sulfonates, naphthalene-2-sulfonates, phenylacetates, phenylpropionates, phenylbutyrates, citrates, lactates, γ-hydroxybutyrates, glycolates, tartrates, and mandelates.

As used herein, the term “therapeutically effective amount” or “effective amount” refers to an amount of a therapeutic agent that when administered alone or in combination with an additional therapeutic agent to a cell, tissue, or subject is effective to prevent or ameliorate a disease or disorder, a proliferation disease or disorder, in a subject. A therapeutically effective dose further refers to that amount of the therapeutic agent sufficient to result in amelioration of symptoms, e.g., treatment, healing, prevention or amelioration of the relevant medical condition, or an increase in rate of treatment, healing, prevention or amelioration of such conditions. When applied to an individual active ingredient administered alone, a therapeutically effective dose refers to that ingredient alone. When applied to a combination, a therapeutically effective dose refers to combined amounts of the active ingredients that result in the therapeutic effect, whether administered in combination, serially or simultaneously. In some embodiment, “an effective amount of a compound for treating a particular disease” is an amount that is sufficient to ameliorate, or in some manner reduce the symptoms associated with the disease. Such amount may be administered as a single dosage or may be administered according to a regimen, whereby it is effective. The amount may cure the disease but, typically, is administered in order to ameliorate the symptoms of the disease. Repeated administration may be required to achieve the desired amelioration of symptoms.

The term “combination” refers to either a fixed combination in one dosage unit form, or a kit of parts for the combined administration where Enzastaurin and an inhibitor of Bruton's tyrosine kinase (BTK) (e.g., another drug as explained below, also referred to as “therapeutic agent” or “co-agent”) may be administered independently at the same time or separately within time intervals, especially where these time intervals allow that the combination partners show a cooperative, e.g., synergistic effect. The terms “co-administration” or “combined administration” or the like as utilized herein are meant to encompass administration of the selected combination partner to a single subject in need thereof (e.g., a patient), and are intended to include treatment regimens in which the agents are not necessarily administered by the same route of administration or at the same time. The term “pharmaceutical combination” as used herein means a product that results from the mixing or combining of more than one active ingredient and includes both fixed and non-fixed combinations of the active ingredients. In some embodiments, the term “fixed combination” means that Enzastaurin and an inhibitor of Bruton's tyrosine kinase (BTK) are both administered to a patient simultaneously in the form of a single entity or dosage. In some embodiments, the term “non-fixed combination” means that Enzastaurin and an inhibitor of Bruton's tyrosine kinase (BTK) are both administered to a patient as separate entities either simultaneously, concurrently or sequentially with no specific time limits, wherein such administration provides therapeutically effective levels of the two substances in the body of the patient. The latter also applies to cocktail therapy, e.g., the administration of three or more active ingredients.

The terms “level” or “levels” are used to refer to the presence and/or amount of a target, e.g., a substance or an organism that is part of the etiology of a disease or disorder, and can be determined qualitatively or quantitatively. A “qualitative” change in the target level refers to the appearance or disappearance of a target that is not detectable or is present in samples obtained from normal controls. A “quantitative” change in the levels of one or more targets refers to a measurable increase or decrease in the target levels when compared to a healthy control.

A “healthy control” or “normal control” is a biological sample taken from an individual who does not suffer from a disease or disorder, e.g., a proliferation disease or disorder,. A “negative control” is a sample that lacks any of the specific analyte the assay is designed to detect and thus provides a reference baseline for the assay.

As used herein, “mammal” refers to any of the mammalian class of species. Frequently, the term “mammal,” as used herein, refers to humans, human subjects or human patients. “Mammal” also refers to any of the non-human mammalian class of species, e.g., experimental, companion or economic non-human mammals. Exemplary non-human mammals include mice, rats, rabbits, cats, dogs, pigs, cattle, sheep, goats, horses, monkeys, Gorillas and chimpanzees.

As used herein, the term “subject” is not limited to a specific species or sample type. For example, the term “subject” may refer to a patient, and frequently a human patient. However, this term is not limited to humans and thus encompasses a variety of non-human animal or mammalian species.

As used herein, a “prodrug” is a substance that, upon in vivo administration, is metabolized or otherwise converted to the biologically, pharmaceutically or therapeutically active form of the substance. To produce a prodrug, the pharmaceutically active substance is modified such that the active substance will be regenerated by metabolic processes. The prodrug may be designed to alter the metabolic stability or the transport characteristics of a drug, to mask side effects or toxicity, to improve the flavor of a drug or to alter other characteristics or properties of a drug. By virtue of knowledge of pharmacodynamic processes and drug metabolism in vivo, those of skill in this art, once a pharmaceutically active compound is known, can design prodrugs of the compound (see, e.g., Nogrady (1985) Medicinal Chemistry A Biochemical Approach, Oxford University Press, New York, pages 388-392).

“Polynucleotide,” or “nucleic acid,” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase. A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may be further modified after polymerization, such as by conjugation with a labeling component. Other types of modifications include, for example, “caps”, substitution of one or more of the naturally occurring nucleotides with an analog, internucleotide modifications such as, for example, those with uncharged linkages (e.g., methyl phosphonates, phosphotriesters, phosphoamidates, cabamates, etc.) and with charged linkages (e.g., phosphorothioates, phosphorodithioates, etc.), those containing pendant moieties, such as, for example, proteins (e.g., nucleases, toxins, antibodies, signal peptides, ply-L-lysine, etc.), those with intercalators (e.g., acridine, psoralen, etc.), those containing chelators (e.g., metals, radioactive metals, boron, oxidative metals, etc.). those containing alkylators, those with modified linkages (e.g., alpha anomeric nucleic acids, etc.), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid supports. The 5′ and 3′ terminal OH can be phosphorylated or substituted with amines or organic capping groups moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-2′-O-allyl, 2′-fluoro- or 2′-azido-ribose, carbocyclic sugar analogs, a-anomeric sugars, epimeric sugars such as arabinose, xyloses or lyxoses, pyranose sugars, furanose sugars, sedoheptuloses, acyclic analogs and abasic nucleoside analogs such as methyl riboside. One or more phosphodiester linkages may be replaced by alternative linking groups. These alternative linking groups include, but are not limited to, embodiments wherein phosphate is replaced by P(O)S(“thioate”), P(S)S (“dithioate”), “(O)NR 2 (“amidate”), P(O)R, P(O)OR′, CO or CH 2 (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

“Oligonucleotide,” as used herein, generally refers to short, generally single stranded, generally synthetic polynucleotides that are generally, but not necessarily, less than about 200 nucleotides in length. The terms “oligonucleotide” and “polynucleotide” are not mutually exclusive. The description above for polynucleotides is equally and fully applicable to oligonucleotides.

As used herein, the term “homologue” is used to refer to a nucleic acid which differs from a naturally occurring nucleic acid (e.g., the “prototype” or “wild-type” nucleic acid) by minor modifications to the naturally occurring nucleic acid, but which maintains the basic nucleotide structure of the naturally occurring form. Such changes include, but are not limited to: changes in one or a few nucleotides, including deletions (e.g., a truncated version of the nucleic acid) insertions and/or substitutions. A homologue can have enhanced, decreased, or substantially similar properties as compared to the naturally occurring nucleic acid. A homologue can be complementary or matched to the naturally occurring nucleic acid. Homologues can be produced using techniques known in the art for the production of nucleic acids including, but not limited to, recombinant DNA techniques, chemical synthesis, etc.

As used herein, “substantially complementary or substantially matched” means that two nucleic acid sequences have at least 90% sequence identity. Preferably, the two nucleic acid sequences have at least 95%, 96%, 97%, 98%, 99% or 100% of sequence identity. Alternatively, “substantially complementary or substantially matched” means that two nucleic acid sequences can hybridize under high stringency condition(s).

In general, the stability of a hybrid is a function of the ion concentration and temperature. Typically, a hybridization reaction is performed under conditions of lower stringency, followed by washes of varying, but higher, stringency. Moderately stringent hybridization refers to conditions that permit a nucleic acid molecule such as a probe to bind a complementary nucleic acid molecule. The hybridized nucleic acid molecules generally have at least 60% identity, including for example at least any of 70%, 75%, 80%, 85%, 90%, or 95% identity. Moderately stringent conditions are conditions equivalent to hybridization in 50% formamide, 5×Denhardt's solution, 5×SSPE, 0.2% SDS at 42° C., followed by washing in 0.2×SSPE, 0.2% SDS, at 42° C. High stringency conditions can be provided, for example, by hybridization in 50% formamide, 5×Denhardt's solution, 5×SSPE, 0.2% SDS at 42° C., followed by washing in 0.1×SSPE, and 0.1% SDS at 65° C. Low stringency hybridization refers to conditions equivalent to hybridization in 10% formamide, 5×Denhardt's solution, 6×SSPE, 0.2% SDS at 22° C., followed by washing in 1×SSPE, 0.2% SDS, at 37° C. Denhardt's solution contains 1% Ficoll, 1% polyvinylpyrolidone, and 1% bovine serum albumin (BSA). 20×SSPE (sodium chloride, sodium phosphate, ethylene diamide tetraacetic acid (EDTA)) contains 3M sodium chloride, 0.2M sodium phosphate, and 0.025 M (EDTA). Other suitable moderate stringency and high stringency hybridization buffers and conditions are well known to those of skill in the art.

As used herein, “vector (or plasmid)” refers to discrete elements that are used to introduce heterologous DNA into cells for either expression or replication thereof. Selection and use of such vehicles are well known within the skill of the artisan. An expression vector includes vectors capable of expressing DNA's that are operatively linked with regulatory sequences, such as promoter regions, that are capable of effecting expression of such DNA fragments. Thus, an expression vector refers to a recombinant DNA or RNA construct, such as a plasmid, a phage, recombinant virus or other vector that, upon introduction into an appropriate host cell, results in expression of the cloned DNA. Appropriate expression vectors are well known to those of skill in the art and include those that are replicable in eukaryotic cells and/or prokaryotic cells and those that remain episomal or those which integrate into the host cell genome.

As used herein, “a promoter region or promoter element” refers to a segment of DNA or RNA that controls transcription of the DNA or RNA to which it is operatively linked. The promoter region includes specific sequences that are sufficient for RNA polymerase recognition, binding and transcription initiation. This portion of the promoter region is referred to as the promoter. In addition, the promoter region includes sequences that modulate this recognition, binding and transcription initiation activity of RNA polymerase. These sequences may be cis acting or may be responsive to trans acting factors. Promoters, depending upon the nature of the regulation, may be constitutive or regulated. Exemplary promoters contemplated for use in prokaryotes include the bacteriophage T7 and T3 promoters, and the like.

As used herein, “operatively linked or operationally associated” refers to the functional relationship of DNA with regulatory and effector sequences of nucleotides, such as promoters, enhancers, transcriptional and translational stop sites, and other signal sequences. For example, operative linkage of DNA to a promoter refers to the physical and functional relationship between the DNA and the promoter such that the transcription of such DNA is initiated from the promoter by an RNA polymerase that specifically recognizes, binds to and transcribes the DNA. In order to optimize expression and/or in vitro transcription, it may be necessary to remove, add or alter 5′ untranslated portions of the clones to eliminate extra, potential inappropriate alternative translation initiation (i.e., start) codons or other sequences that may interfere with or reduce expression, either at the level of transcription or translation. Alternatively, consensus sites can be inserted immediately 5′ of the start codon and may enhance expression. See, e.g., Kozak (1991) J. Biol. Chem. 266:19867-19870. The desirability of (or need for) such modification may be empirically determined.

As used herein, “biological sample” refers to any sample obtained from a living or viral source or other source of macromolecules and biomolecules, and includes any cell type or tissue of a subject from which nucleic acid or protein or other macromolecule can be obtained. The biological sample can be a sample obtained directly from a biological source or a sample that is processed. For example, isolated nucleic acids that are amplified constitute a biological sample. Biological samples include, but are not limited to, body fluids, such as blood, plasma, serum, cerebrospinal fluid, synovial fluid, urine and sweat, tissue and organ samples from animals and plants and processed samples derived therefrom.

As used herein, “production by recombinant means” refers to production methods that use recombinant nucleic acid methods that rely on well-known methods of molecular biology for expressing polypeptides or proteins encoded by cloned nucleic acids.

It is understood that aspects and embodiments of the invention described herein include “consisting of” and/or “consisting essentially of” aspects and embodiments.

Throughout this disclosure, various aspects of this invention are presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Other objects, advantages and features of the present invention will become apparent from the following specification taken in conjunction with the accompanying drawings.

### Exemplary Embodiments

In some embodiments, the present invention is illustrated by the following enumerated embodiments:

1. A composition comprising enzastaurin, or a pharmaceutically acceptable salt thereof, and an inhibitor of BTK. In some of these embodiments, enzastaurin is present as its hydrochloride salt.

2. The composition of embodiment 1, wherein the inhibitor of BTK is selected from M7583, ibrutinib, acalabrutinib, zanubrutinib, CT-1530, DTRMWXHS-12, spebrutinib besylate, vecabrutinib, evobrutinib, tirabrutinib, fenebrutinib, poseltinib, BMS -986142, ARQ-531,LOU-064, PRN-1008, ABBV-599, AC-058, ARQ-531, BIIB-068, BMS-986195, HWH-486, PRN-2246, TAK-020, GDC-0834, BMX-IN-1, RN486, SNS-062, LFM-A13, PCI-32765 (racemate of ibrutinib), CGI-1746, ONO-4059, and SHR-1459, and their pharmaceutically acceptable salts, and preferably the inhibitor of BTK is selected from M7583, ibrutinib, acalabrutinib, zanubrutinib, CT-1530, DTRMWXHS-12, spebrutinib besylate, vecabrutinib, ARQ-531, and SHR-1459, and their pharmaceutically acceptable salts.

3. The composition of embodiment 1 or 2, wherein the inhibitor of BTK is selected from M7583, Ibrutinib, and Acalabrutinib and the pharmaceutically acceptable salts thereof, and is preferably ibrutinib or a pharmaceutically acceptable salt thereof.

4. The composition of any of embodiments 1-3, which further comprises at least one pharmaceutically acceptable carrier or excipient. Optionally, the composition comprises two or more pharmaceutically acceptable carriers or excipients.

5. A therapeutic combination, comprising enzastaurin, or a pharmaceutically acceptable salt thereof, and an inhibitor of BTK.

6. The therapeutic combination of embodiment 5, wherein the inhibitor of BTK is selected from M7583, ibrutinib, acalabrutinib, zanubrutinib, CT-1530, DTRMWXHS-12, spebrutinib besylate, vecabrutinib, evobrutinib, tirabrutinib, fenebrutinib, poseltinib, BMS-986142, ARQ-531,LOU-064, PRN-1008, ABBV-599, AC-058, ARQ-531, BIIB-068, BMS-986195, HWH-486, PRN-2246, TAK-020, GDC-0834, BMX-IN-1, RN486, SNS-062, LFM-A13, PCI-32765 (racemate of ibrutinib), CGI-1746, ONO-4059, and SHR-1459, and their pharmaceutically acceptable salts, and preferably the BTK inhibitor is selected from M7583, ibrutinib, acalabrutinib, zanubrutinib, CT-1530, DTRMWXHS-12, spebrutinib besylate, vecabrutinib, ARQ-531, and SHR-1459, and the pharmaceutically acceptable salts thereof.

7. The therapeutic combination of embodiment 5 or 6, wherein the inhibitor of BTK is selected from M7583, Ibrutinib, and Acalabrutinib, or a pharmaceutically acceptable salt thereof; and preferably the inhibitor of BTK is ibrutinib or a pharmaceutically acceptable salt thereof.

8. The therapeutic combination of any one of embodiments 5-7, wherein enzastaurin and the inhibitor of BTK are prepared for simultaneous administration.

9. The therapeutic combination of any one of embodiments 5-7, wherein enzastaurin, or a pharmaceutically acceptable salt thereof, and the BTK inhibitor are prepared for separate administration.

10. An in vivo therapeutic combination, which comprises enzastaurin and a BTK inhibitor in the blood or plasma of a subject.

11. A method to treat a disorder or disease selected from oncologic conditions, immunological disorders, gastrointestinal disorders, CNS disorders, dermatological disorders, hematological disorders, and metabolic disorders, wherein the method comprises administering to a subject in need of such treatment enzastaurin and an inhibitor of BTK. In these methods, the subject is typically a human and is optionally a human diagnosed with lymphoma. In some of these embodiments, enzastaurin is used as its hydrochloride salt. In some embodiments, an effective amount of enzastaurin and/or of the BTK inhibitor is administered. In a preferred embodiment, a synergistic amount of enzastaurin and of the BTK inhibitor is administered, i.e., the amounts of enzastaurin and of a BTK inhibitor are sufficient to provide a synergistic effect

12. The method of embodiment 11, which is a method for treatment of a cancer selected from chronic lymphocytic leukemia (CLL), extranodal marginal zone B-cell lymphoma, mucosa-associated lymphoid tissue lymphoma (MALT-lymphoma), Waldenstrom's macroglobulinemia, mantle cell lymphoma, relapsed CLL, refractory CLL, follicular lymphoma, adenocarcinoma, metastatic adenocarcinoma (e.g., pancreatic), non-Hodgkin lymphoma, pancreatic cancer, acute lymphocytic leukemia, acute lymphoblastic leukemia, hairy cell leukemia, metastatic breast cancer, acute myelocytic leukemia, acute myeloblastic leukemia, multiple meloma, refractory multiple myeloma, relapsed multiple myeloma, gastric cancer, colorectal cancer, bladder cancer, Hodgkin lymphoma (B-cell Hodgkin lymphoma), metastic melanoma, non-small cell lung cancer, primary CNS lymphoma, renal cell carcinoma, secondary CNS lymphoma, transitional cell carcinoma, urothelial cell carcinoma, nodal marginal B-cell lymphoma, splenic marginal zone B-cell lymphoma, T-cell lymphomas, epithelial ovarian cancer, fallopian tube cancer, peritoneal cancer, (recurrent) head and neck cancer, squamous cell carcinoma, (recurrent) glioblastoma multiforme (GBM), and B-Cell lymphoma including diffuse large B-cell lymphoma.

13. The method of embodiment 12, wherein the inhibitor of BTK is selected from M7583, ibrutinib, acalabrutinib, zanubrutinib, CT-1530, DTRMWXHS-12, spebrutinib besylate, vecabrutinib, ARQ-531, and SHR-1459, or a pharmaceutically acceptable salt thereof. In some such embodiments the inhibitor of BTK is ibrutinib or a pharmaceutically acceptable salt thereof.

14. A method for treatment or prevention of lymphoma, or for reduction of risk of metastasis or relapse in a subject having been treated for lymphoma, wherein the method comprises administering to a subject in need thereof, enzastaurin, or a pharmaceutically acceptable salt thereof, and an inhibitor of BTK.

15. The method of embodiment 14, wherein the inhibitor of BTK is selected from M7583, ibrutinib, acalabrutinib, zanubrutinib, CT-1530, DTRMWXHS-12, spebrutinib besylate, vecabrutinib, evobrutinib, tirabrutinib, fenebrutinib, poseltinib, BMS-986142, ARQ-531, LOU-064, PRN-1008, ABBV-599, AC-058, ARQ-531, BIIB-068, BMS-986195, HWH-486, PRN-2246, TAK-020, GDC-0834, BMX-IN-1, RN486, SNS-062, LFM-A13, PCI-32765 (racemate of ibrutinib), CGI-1746, ONO-4059, and SHR-1459, and their pharmaceutically acceptable salts, and preferably the BTK inhibitor is selected from M7583, ibrutinib, acalabrutinib, zanubrutinib, CT-1530, DTRMWXHS-12, spebrutinib besylate, vecabrutinib, ARQ-531, and SHR-1459, and the pharmaceutically acceptable salts thereof

16. The method of embodiment 15, wherein the inhibitor of BTK is selected from M7583, ibrutinib, acalabrutinib, zanubrutinib, CT-1530, DTRMWXHS-12, spebrutinib besylate, vecabrutinib, ARQ-531, and SHR-1459 and their pharmaceutically acceptable salts, and preferably the inhibitor of BTK is ibrutinib or a pharmaceutically acceptable salt thereof.

17. The method of embodiment 14, 15 or 16, wherein the inhibitor of BTK is selected from M7583, Ibrutinib, and Acalabrutinib, or a pharmaceutically acceptable salt thereof.

18. The method of any one of embodiments 11-17, wherein enzastaurin, or a pharmaceutically acceptable salt thereof, and the inhibitor of BTK are administered together. In these embodiments, enzastaurin or a pharmaceutically acceptable salt thereof is optionally co-formulated with a BTK inhibitor as a single pharmaceutical composition. In other of these embodiments, enzastaurin or a pharmaceutically acceptable salt thereof and the BTK inhibitor are in separate pharmaceutical compositions, but are administered at about the same time, i.e. they are taken separately but within a matter of minutes or within about an hour, rather than being spaced apart by more than an hour.

19. The method of any one of embodiments 11-17, wherein enzastaurin, or a pharmaceutically acceptable salt thereof, and the inhibitor of BTK are administered separately. In these embodiments, enzastaurin or a pharmaceutically acceptable salt thereof and a BTK inhibitor are in separate pharmaceutical compositions, and may be administered at about the same time, i.e. they may be taken separately but within a matter of minutes, or they may be administered at different times, such as being spaced apart by an hour or more in time, or by an intervening meal or other event, but both are administered within a 24 hour period, or within a 48 hr period.

20. The method of embodiment 19, wherein enzastaurin, or a pharmaceutically acceptable salt thereof, and the inhibitor of BTK are administered on a schedule which causes both to be present in the blood or plasma of the treated subject together. In these embodiments, enzastaurin and the BTK inhibitor are administered closely enough in time to cause both to be present in the blood or plasma of the treated subject at measurable levels, typically at a level of at least 5%, and typically at least 10%, of the Cmax for each of the two individual agents.

21. The method of any one of embodiments 11-20, wherein lymphoma is Hodgkin lymphoma or Non-Hodgkin lymphoma.

22. The method of embodiment 21, wherein lymphoma is non-Hodgkin lymphoma.

23. The method of embodiment 22, wherein the lymphoma is selected from Burkitt's lymphoma, small lymphocytic lymphoma, a B-cell lymphoma, lymphoplasmacytic lymphoma, extranodal marginal zone B cell lymphoma, follicular lymphoma, diffuse large B-cell lymphoma, mantle cell lymphoma, mycosis fungoides, small lymphocytic lymphoma, and anaplastic large cell lymphoma. In a preferred version of this embodiment, the lymphoma is diffuse large B-cell lymphoma.

24. The method of any one of embodiments 11-23, wherein enzastaurin, or a pharmaceutically acceptable salt thereof, is administered orally. In some of these embodiments, enzastaurin is administered as its hydrochloride salt. In some of these embodiments, the amount of enzastaurin or enzastaurin hydrochloride administered to the subject is 500 mg per day, or less than 500 mg per day.

25. The method of embodiment 24, wherein the inhibitor of BTK is administered orally.

26. The method of any one of embodiments 11-25, wherein the inhibitor of BTK is ibrutinib or a pharmaceutically acceptable salt thereof. In some of these embodiments the dosage of the BTK inhibitor is 400 mg per day, or less than 400 mg per day. In some embodiments, the ratio of enzastaurin to BTK inhibitor by weight, particularly where the BTK inhibitor is ibrutinib, is 1:1 or greater, e.g., 2:1, 3:1, 4:1, 5:1, 6:1, or 8:1.

27. The method of any of embodiments 11-26, wherein the subject is selected based on expression or presence of a biomarker.

28. The method of embodiment 26, wherein the biomarker is DGM1 .

Reference to enzastaurin or ibrutinib throughout is intended to include the neutral compound or a pharmaceutically acceptable salts of each compound. In particular, enzastaurin can be prepared, formulated or used as a neutral molecule or as its hydrochloride salt. Ibrutinib can be prepared, formulated or used as any suitable acid-addition product, including salts and solid forms disclosed in International Application No. PCT/EP2016/056312 and PCT/EP2015/069430. Preferably, ibrutinib is used as the neutral compound. Weights and dosages disclosed herein refer to the neutral compounds, e.g., when an amount refers to 500 mg of enzastaurin or a pharmaceutically acceptable salt thereof, the weight is intended to describe the weight of neutral enzastaurin to be used for consistency, regardless of which salt is used, if any. Unless otherwise indicated, the weights of enzastaurin or BTK inhibitor include a range of ±10% of the specified quantity.

The compounds and compositions described herein can be administered to a subject in need of treatment for a cell proliferation disorder such as cancer, particularly cancers that respond to treatment with an inhibitor of BTK, or for treatment of other indications disclosed herein.

In some embodiments, the disorder is a cancer selected from leukemia, lymphoma, lung cancer, colon cancer, CNS cancer, melanoma, ovarian cancer, renal cancer, prostate cancer, breast cancer, head and neck cancers, and pancreatic cancer. The subject is typically a mammal diagnosed as being in need of treatment for one or more of such proliferative disorders, and frequently the subject is a human. The methods comprise administering an effective amount of at least one compound of the combination, i.e., enzastaurin or a BTK inhibitor, and an amount that may also be an effective amount of the other compound. In some embodiments, the two components are administered in an amount or in a proportion or ratio that provides synergistic activity, so the combination administered is an effective amount, even if the separately administered individual agents would not be expected to provide a therapeutic effect when used at that amount; thus ‘effective’ in the context of the combinations includes synergistic effects. Optionally the therapeutic combination or pharmaceutical composition may be administered in combination with one or more additional therapeutic agents, particularly therapeutic agents known to be useful for treating the cancer or proliferative disorder afflicting the particular subject, and/or a PD-1 or PD-L1 antagonist.

In addition to the combination of the invention, or prior to its use, the subject may be treated with other therapeutic agents indicated for the particular condition to be treated. Commonly, subjects having lymphoma are treated with rituximab and/or doxorubicin in combination with other approved therapeutics. Conventional chemotherapy combinations that may be used in combination with or prior to treatment with enzastaurin plus a BTK inhibitor (such as ibrutinib) include CHOP and R-CHOP. CHOP is an acronym for a treatment regimen that includes cyclophosphamide, hydroxydaunorubicin (doxorubicin hydrochloride), Oncovin (vincristine sulfate), and prednisone. R-CHOP is an abbreviation for a chemotherapy combination that is used to treat non-Hodgkin lymphoma and mantle cell lymphoma and is being studied in the treatment of other types of cancer. It includes the drugs rituximab, cyclophosphamide, doxorubicin hydrochloride (hydroxydaunorubicin), vincristine sulfate (Oncovin), and prednisone.

In some embodiments, the invention provides a method to protect a subject from metastasis or relapse after the subject has been treated by any suitable method for lymphoma such as DLBCL, where the method comprises administering to a subject in need of such protection enzastaurin and a BTK inhibitor such as ibrutinib.

In each of the foregoing methods, the method can be used to treat a subject having lymphoma such as DLBCL, and in some methods the subject is one having already been treated by at least one other method such as CHOP, R-CHOP, or the like, and still experienced progression. In other embodiments the subject is one who has been treated by these methods and achieved at least partial response, in which case the subject may be treated with enzastaurin and a BTK inhibitor such as ibrutinib to protect the subject from relapse or metastasis. Moreover, in some embodiments the subject s selected based on a biomarker response, for example a subject may be deemed suitable for treatment with the compositions and therapeutic combinations disclosed herein when the subject is selected based on expression or presence of a biomarker, particularly

### Pharmaceutical Compositions, Combinations, and other Related Uses

In still another aspect, the present disclosure provides for a pharmaceutical composition comprising a combination of enzastaurin and a BTK inhibitor as described herein, admixed with at least one pharmaceutically acceptable carrier or excipient. Optionally, the pharmaceutical composition comprises at least two pharmaceutically acceptable carriers or excipients. Suitable excipients and carriers for use in pharmaceutical compositions of these compounds are known in the art.

The above described combinations and compositions can be used for any suitable purpose. For example, they can be used in therapy and/or testing. Typically, they are used to treat a subject in need of treatment for a B-cell disorder, particularly a B-cell cancer such as lymphoma.

In yet another aspect, the present disclosure provides for a use of a combination as described above for the manufacture of a medicament.

In one aspect, the invention provides a combination of enzastaurin and a BTK inhibitor such as ibrutinib for use in therapy.

In some embodiments, the combination is for use in therapy for treating a form of lymphoma, such as DLBCL.

In yet another aspect, the present disclosure provides a method for inhibiting an activity of BTK in a cell, organ or tissue, which comprises contacting the cell, organ or tissue with a combination of enzastaurin and a BTK inhibitor, preferably ibrutinib.

### Formulations

Any suitable formulation of the compounds described herein can be used. See generally, Remington's Pharmaceutical Sciences, (2000) Hoover, J. E. editor, 20th edition, Lippincott Williams and Wilkins Publishing Company, Easton, Pa., pages 780-857. A formulation is selected to be suitable for an appropriate route of administration. Viable formulations of enzastaurin are known and can be used as information for design of a new formulation such as a combination with a BTK inhibitor. Similarly, safe and effective formulations of some BTK inhibitors, including ibrutinib, are known and can be used for the present invention or modified as needed, such as for use in a pharmaceutical composition that also contains enzastaurin.

In cases where compounds are sufficiently basic or acidic to form stable nontoxic acid or base salts, administration of the compounds as salts may be appropriate. Examples of pharmaceutically acceptable salts are organic acid addition salts formed with acids that form a physiological acceptable anion, for example, tosylate, methanesulfonate, acetate, citrate, malonate, tartarate, succinate, benzoate, ascorbate, α-ketoglutarate, and α-glycerophosphate. Suitable inorganic salts may also be formed, including hydrochloride, sulfate, nitrate, bicarbonate, and carbonate salts. Pharmaceutically acceptable salts are obtained using standard procedures well known in the art, for example, by a sufficiently basic compound such as an amine with a suitable acid, affording a physiologically acceptable anion. Alkali metal (e.g., sodium, potassium or lithium) or alkaline earth metal (e.g., calcium) salts of carboxylic acids also are made. In the case of enzastaurin, a known hydrochloride salt can be used in the compositions, combinations and methods disclosed herein.

Where contemplated compounds are administered in a pharmacological composition, it is contemplated that the compounds can be formulated in admixture with a pharmaceutically acceptable excipient and/or carrier. For example, contemplated compounds can be administered orally as neutral compounds or as pharmaceutically acceptable salts, or intravenously in a physiological saline solution. Conventional buffers such as phosphates, bicarbonates or citrates can be used for this purpose. Of course, one of ordinary skill in the art may modify the formulations within the teachings of the specification to provide numerous formulations for a particular route of administration. In particular, contemplated compounds may be modified to render them more soluble in water or other vehicle, which for example, may be easily accomplished with minor modifications (salt formulation, esterification, etc.) that are well within the ordinary skill in the art. It is also well within the ordinary skill of the art to modify the route of administration and dosage regimen of a particular compound in order to manage the pharmacokinetics of the present compounds for maximum beneficial effect in a patient.

The compounds described herein are generally soluble in organic solvents such as chloroform, dichloromethane, ethyl acetate, ethanol, methanol, isopropanol, acetonitrile, glycerol, N,N-dimethylformamide, N,N-dimetheylaceatmide, dimethylsulfoxide, etc. In one embodiment, the present invention provides formulations prepared by mixing the combination of enzastaurin and a BTK inhibitor with a pharmaceutically acceptable carrier. In one aspect, the formulation may be prepared using a method comprising: a) dissolving the selected compound(s) in a water-soluble organic solvent, a non-ionic solvent, a water-soluble lipid, a cyclodextrin, a vitamin such as tocopherol, a fatty acid, a fatty acid ester, a phospholipid, or a combination thereof, to provide a solution; and b) adding saline or a buffer containing 1-10% carbohydrate solution. In one example, the carbohydrate comprises dextrose. The pharmaceutical compositions obtained using the present methods are stable and useful for animal and clinical applications.

Illustrative examples of water soluble organic solvents for use in the present methods include and are not limited to polyethylene glycol (PEG), alcohols, acetonitrile, N-methyl-2-pyrrolidone, N,N-dimethylformamide, N,N-dimethylacetamide, dimethyl sulfoxide, or a combination thereof. Examples of alcohols include but are not limited to methanol, ethanol, isopropanol, glycerol, or propylene glycol.

Illustrative examples of water soluble non-ionic surfactants for use in the present methods include and are not limited to CREMOPHOR® EL, polyethylene glycol modified CREMOPHOR® (polyoxyethyleneglyceroltriricinoleat 35), hydrogenated CREMOPHOR® RH40, hydrogenated CREMOPHOR® RH60, PEG-succinate, polysorbate 20, polysorbate 80, SOLUTOL® HS (polyethylene glycol 660 12-hydroxystearate), sorbitan monooleate, poloxamer, LABRAFIL® (ethoxylated persic oil), LABRASOL® (capryl-caproyl macrogol-8-glyceride), GELUCIRE® (glycerol ester), SOFTIGEN® (PEG 6 caprylic glyceride), glycerin, glycol-polysorbate, or a combination thereof.

Illustrative examples of water soluble lipids for use in the present methods include but are not limited to vegetable oils, triglycerides, plant oils, or a combination thereof. Examples of lipid oils include but are not limited to castor oil, polyoxyl castor oil, corn oil, olive oil, cottonseed oil, peanut oil, peppermint oil, safflower oil, sesame oil, soybean oil, hydrogenated vegetable oil, hydrogenated soybean oil, a triglyceride of coconut oil, palm seed oil, and hydrogenated forms thereof, or a combination thereof.

Illustrative examples of fatty acids and fatty acid esters for use in the present methods include but are not limited to oleic acid, monoglycerides, diglycerides, a mono- or di-fatty acid ester of PEG, or a combination thereof.

Illustrative examples of cyclodextrins for use in the present methods include but are not limited to alpha-cyclodextrin, beta-cyclodextrin, hydroxypropyl-beta-cyclodextrin, or sulfobutyl ether-beta-cyclodextrin.

Illustrative examples of phospholipids for use in the present methods include but are not limited to soy phosphatidylcholine, or distearoyl phosphatidylglycerol, and hydrogenated forms thereof, or a combination thereof.

In some embodiments, enzastaurin and the BTK inhibitor of choice (e.g., ibrutinib) are combined in a single pharmaceutical composition, typically with one or more pharmaceutically acceptable carriers or excipients. Suitable carriers and excipients for each of the separate compounds are known in the art, and carriers and excipients for the combination can be selected from those known to be suitable for the separate formulations. In these embodiments, the proportion of enzastaurin to the BTK inhibitor in a pharmaceutical composition can be selected based on information known in the art, and typically ranges from about 5:1 to about 1:2, depending upon the BTK inhibitor. Unit dosage size can similarly be determined based on data herein in combination with information such as clinical trials of the separate active ingredients. Information herein can provide further guidance, as it demonstrates that a synergistic effect is expected for the combination.

In some embodiments, enzastaurin and the BTK inhibitor are formulated separately. Suitable formulations for each of these compounds are known in the art.

Preferably, the pharmaceutical compositions and combinations of the invention are prepared for oral administration, as a pill, lozenge, troche, capsule, or similar solid dosage form. The components may be combined into a single composition, but in some embodiments, they are prepared as separate unit dosages instead of being combined into a single composition or unit dosage. This provides maximum flexibility in optimizing the combination for a particular patient, so the administration frequency, timing and dosage of each component can best be optimized for a particular subject being treated. Solid dosage forms of both enzastaurin and BTK inhibitors such as ibrutinib are known in the art: in some embodiments of the invention, the known solid dosage forms are administered to a subject, and dosages are established using guidelines for the individual therapeutic agents being administered.

When prepared for separate administration, the components of the therapeutic combinations of the invention may be contained in separate dosage units, e.g. enzastaurin and the BTK inhibitor chosen, such as ibrutinib, may be in different pills, capsules, troches, lozenges, or suspensions. Where the two active components of the combination are not admixed, the separate dosage units can be packaged together such as in a blister pack for co-administration, and either or both of the two actives (e.g., enzastaurin and a BTK inhibitor, which can be ibrutinib) when in separate dosage units can be packaged with instructions for using the enzastaurin composition with a BTK inhibitor composition, or vice versa. Thus in some embodiments, the therapeutic combination can comprise a pharmaceutical composition comprising either enzastaurin or ibrutinib (or another chosen BTK inhibitor) packaged with instructions for use according to the methods herein for administering enzastaurin and a BTK inhibitor to a subject in need of treatment for a B-cell lymphatic cancer such as lymphoma, and in particular DLBCL. In some embodiments, the therapeutic combination may be a kit that comprises an effective amount of enzastaurin and ibrutinib, whether formulated as a single composition or as separate unit dosages, with instructions for administering enzastaurin and a BTK inhibitor to a subject in need of treatment fora B-cell lymphatic cancer such as lymphoma, and in particular DLBCL.

### Dosages

In some embodiments, the individual components are administered at the low end of the range of normal dosages for use as single-agent therapeutics, or at lower dosage, i.e. they can be administered at the lowest dosage expected to be effective as a single-agent therapy, or at a lower dosage. Thus, the subject may be treated with a daily dosage containing less of the active agent (enzastaurin, or ibrutinib for example) than a daily dosage intended to produce a therapeutic effect. Thus, a subject may be treated with fewer unit dosages per day than would be administered to elicit a therapeutic effect, or the unit dosages of one or both of the active agents may be administered less frequently than they would be when intended to elicit a therapeutic effect as a single agent. A ‘unit dosage’ as used herein refers to a dose of a therapeutic agent or combination prepared as the smallest unit intended for administration to a subject, e.g., a single ampoule for injection, or a single tablet or capsule for oral administration. It is understood that a single dose may be comprised of two or more of such unit dosages, and that a daily dosage may be taken all at one time or in multiple doses such as two or three separate administrations spaced apart by two hours or more over the course of a day.

Also, due to the synergistic activity provided by combining enzastaurin with a BTK inhibitor, the individual components of the combination can be administered less frequently than they would be when administered as single agent therapies, in order to produce plasma concentrations below those targeted for single-agent therapeutic effects. In some embodiments, at least one of the two components of the combination is administered at a dosage that would not be expected to achieve a therapeutic result if used alone, e.g. at about 90% or less than 90% of a single-agent dosage, or at half of the dosage that would be used for single-agent therapy, or less than half of that dosage.

Ibrutinib, for example is approved in the U.S. for treating certain B-cell cancers (mantle cell lymphoma, CLL, small lymphocytic lymphoma, Waldenstrom's macroglobulinemia, marginal zone lymphoma) and is available as both capsules (70 mg and 140 mg) and tablets (140 mg, 280 mg, 420 mg and 560 mg). Depending on condition to be treated, the recommended dosage is either 420 mg or 560 mg, once daily. See Imbruvica® prescribing information. When used in combination with enzastaurin, ibrutinib can be taken at dosages lower than 400 mg per day: in some embodiments the daily dosage of ibrutinib for use in the compositions, combinations and methods herein, can be 70 mg, 140 mg, 210 mg, 280 mg, or 350 mg.

Enzastaurin is in a clinical trial for use in combination with a powerful chemotherapy regimen known as R-CHOP (which includes rituximab, cyclophosphamide, doxorubicin, vincristine, and prednisone), and the starting dosage for enzastaurin is 500 mg daily, the same dosage used in some earlier cancer trials in which enzastaurin did not provide a significant therapeutic benefit. When used in combination with a BTK inhibitor, for the compositions, combinations and methods of the present invention, enzastaurin can be administered at a daily dosage of 500 mg, or less than 500 mg, e.g. 250 mg, 300 mg, 350 mg, 400 mg, or 450 mg per day.

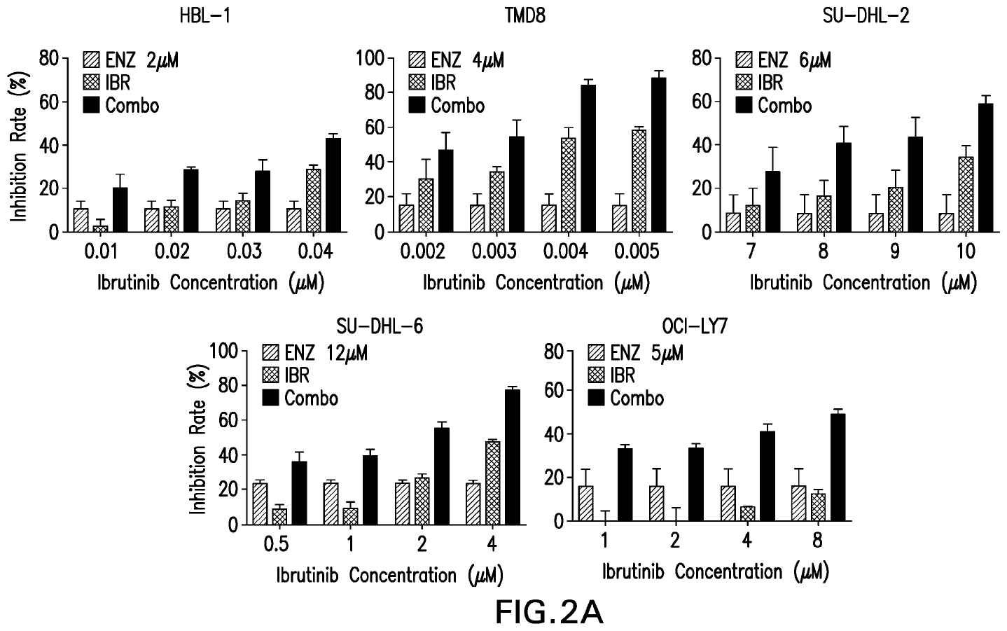

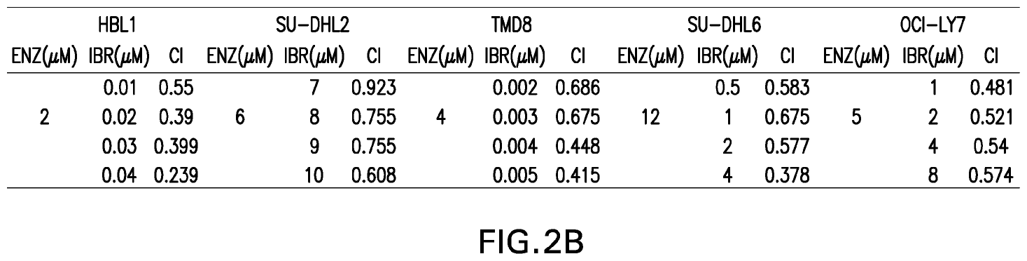

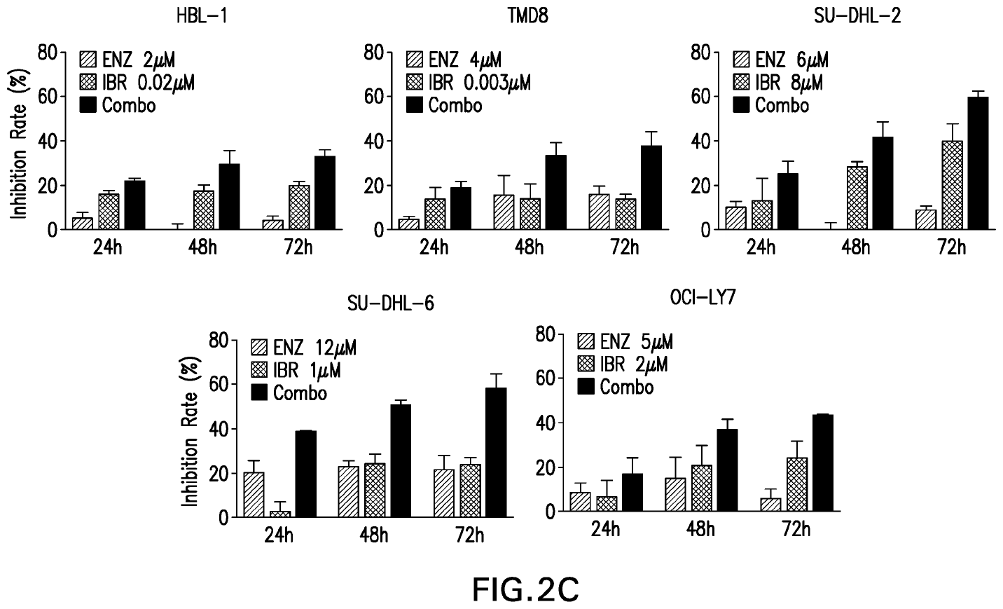

Preferably, the two components of a combination within the scope of the invention, enzastaurin and the BTK inhibitor (e.g., ibrutinib), are administered at a dosage that produces a synergistic effect. As the in vitro data demonstrates, this combination provides synergy across a range of concentrations and proportions. For example, as shown by data summarized in FIG. 2, concentrations of 2-12 μM enzastaurin produced synergistic activity on five different DLBCL cell lines when combined with ibrutinib at concentrations ranging from 0.002 μM to 8 μM. Indeed, the index for synergy (CI) was less than 1 in all of the concentrations and cell lines tested, which is indicative of synergy, and was less than 0.8 in all but one case (CI=0.923 for the SU-DHL2 cell line at 6 μM enzastaurin combined with 7 μM ibrutinib). Accordingly, synergy is expected when these two therapeutic agents are used together for treating DBCLC. Moreover, synergy was observed when enzastaurin and ibrutinib were used to treat DBCLC in vivo (mouse xenograft), when enzastaurin and ibrutinib were administered daily, orally, in a proportion of 100 to 12. Thus, synergy is expected to be observed when the ratio of enzastaurin to BTK inhibitor by weight, particularly where the BTK inhibitor is ibrutinib, is 1:1 or greater, e.g., 2:1, 3:1, 4:1, 5:1, 6:1, or 8:1.

As an example, enzastaurin may be administered at a daily dosage of 100 mg, 150 mg, 200 mg, 250 mg, 300 mg, 400 mg, 500 mg, 525 mg or 700 mg to a subject to be treated, and the dosage may be administered in a single dose or it may be divided into two, three, or more than three doses over the course of the day. In some embodiments, enzastaurin is administered once daily or twice daily. Ibrutinib can be administered at the same daily dosage or a lower daily dosage compared to the enzastaurin dosage, and is typically administered in a single daily dose.

Ibrutinib is commonly administered in the form of capsules, at a daily dosage of between 100 and 1000 mg/day, often as a single daily dose of 150 mg, 200 mg, 250 mg, 300 mg, 350 mg, 400 mg, 420 mg or 560 mg once daily, though the dose and frequency of administration may be optimized by the treating physician for a particular subject. Moreover, as discussed herein, synergistic efficacy is observed when enzastaurin and a BTK inhibitor like ibrutinib are used in combination, thus it may be suitable to administer the two agents at lower doses than those typically recommended when each agent is used as a single agent for therapy.

Where the two therapeutic agents in the combination of the invention are administered separately, they may be administered on the same dosing schedule (as separate dosage units taken at about the same time) or on different dosing schedules, provided they are administered in a manner that causes both to be present in the system of the subject concurrently, i.e., each is administered on the same day as the other, typically within 12 hours or less, or within 4 hours or less, or they are each administered closely enough in time to the other to cause both compounds (enzastaurin and the BTK inhibitor) to be present concurrently at levels of at least about 10% of their respective maximum blood or plasma levels (Cmax).

One of ordinary skill in the art may modify the formulations within the teachings of the specification to provide numerous formulations for a particular route of administration. In particular, the compounds may be modified to render them more soluble in water or other vehicle. It is also well within the ordinary skill of the art to modify the route of administration and dosage regimen of a particular compound in order to manage the pharmacokinetics of the present compounds for maximum beneficial effect in a patient.

### Drug combinations

The methods of the embodiments comprise administering an effective amount of enzastaurin and at least one compound known to inhibit BTK; optionally the combination may be administered in combination with one or more additional therapeutic agents, particularly therapeutic agents known to be useful for treating the lymphatic proliferation disorder to be treated with the combination of the invention.

The additional active ingredient(s) may be administered in a separate pharmaceutical composition from the combination of the present disclosure or may be included with at least one compound of the present combination in a single pharmaceutical composition. The additional active ingredient(s) may be administered simultaneously with, prior to, or after administration of at least one exemplary compound of the present disclosure.

### EXAMPLES

The following examples are provided to illustrate certain aspects of the invention and to aid in its practice; they are not to be viewed as the full extent of the invention or as a limitation of its scope.

### Example 1

Material and Methods

Cell Lines and Cell Culture

HBL-1, TMD8, OCI-LY7 cell lines were generously provided by Dr. Fu, University of Nebraska Medical Center (Omaha, Nebr., USA). SU-DHL-2 and SU-DHL-6 cells were obtained from American Type Culture Collection (Manassas, Va.). Cells were grown in RPMI1640 medium (Gibco, Life Technologies, CA, USA) supplemented with 10-20% fetal bovine serum (Gibco, Life Technology, CA, USA), penicillin/streptomycin, glutamine, beta-mercaptoethanol. Except for OCI-Ly7, which was maintained in Iscove's Modified Dulbecco's Medium (IMDM) (Gibco, Life Technology, CA, USA) supplemented with beta-mercaptoethanol, penicillin/streptomycin, and 20% heparinized human plasma. All cell lines were maintained in a humidified 5% CO₂ incubator at 37° C. Identification of all DLBCL cell lines was confirmed by short tandem repeat DNA fmgerprinting analysis (Applied Bio systems, Foster City, Calif., USA).

Drugs and Reagents

Enzastaurin was a gift from Denovo Biopharma (San Diego, USA) and ibrutinib was purchased from Medchem Express (NJ, USA). It was initially dissolved in 100% dimethylsulfoxide (DMSO, Sigma Chemical) at a concentration of 10 mM and stored at −80° C. Primary and secondary antibodies were listed in additional file 1.

Analysis of Cell Proliferation

Cells were seeded in a 96-well culture plate at a density of 3000 cells per 100 μl and treated with different concentrations of enzastaurin and ibrutinib for 72 hours. Cells were counted and viability was assessed using Cell Titer-Glo Luminescent Cell viability assay system (Promega, Madison, Wis., USA). Luminescent signals were measured by LMax II (Molecular Devices, Sunnyvale, Calif., USA). Inhibition rates were calculated following the formula: Inhibition rates=(1-dosing/vehicle)×100%.

Apoptotic Cells and Cell-Cycle Assays

Cells were treated with vehicle or indicated concentrations of enzastaurin and ibrutinib for 48 h for apoptosis and cell cycle analysis. For apoptosis assays, cells were stained with annexin V-APC (Biolegend, CA, USA) according to the protocol. For cell cycle assays, cells were stained with PI staining buffer (Sigma-Aldrich, Darmstadt, Germany) according to the manufacturer's protocol. Finally, the labeled cells were analyzed using BD Accuri C6 flow cytometer (BD, Biosciences, San Jose, Calif.).

Real-Time Reverse Transcription-PCR (qRT-PCR) Assay

Total cellular RNA was extracted using Trizol reagent (Life Technologies, Carlsbad, Calif.) and cDNA was synthesized using TransScript First-Strand cDNA Synthesis SuperMix (TransGen Biotech, Beijing, China). qRT-PCR analysis was performed using Go Taq qPCR Master Mix (Promega Corporation, Madison, USA). Specific primers for NOTCH1 (Forward: 5′-TCCACCAGTTTGAATGGTCAAT-3′ (SEQ ID NO:1); Reverse: 5′-CGCAGAGGGITG TAITGGITC-3′ (SEQ ID NO:2)) and GAPDH (Forward: 5′-GCACCGTCAAGGCTGAGAAC-3′ (SEQ ID NO:3); Reverse: 5′-TGGTGAAGACGC CAGTGGA-3′ (SEQ ID NO:4)) were used to perform qRT-PCR. All reactions were run in Applied Biosystems 7500 Fast Real-Time PCR System (Applied Biosystems, Woburn, Mass., USA), mRNA expression data were calculated using the following equation: RQ=2-ΔΔCt.

Western Blotting and Signaling Assays

Harvested cultured cells were lysed in RIPA buffer (Cell Signaling Technology, Danvers, Mass.) with protease/phosphatase inhibitor (Roche, Mannheim, Germany). Signaling proteins were detected by western blot as previously described [25]. Immunopositive bands were visualized using chemiluminescence detection system (Alpha Innotech, San Leandro, Calif., USA) according to the manufacturer's instructions.

Invasion and Migration Assay

Cells were treated with vehicle or indicated concentrations of enzastaurin and ibrutinib for indicated time in FBS-free RPMI1640. For cell invasion assays, cells were placed into Matrigel basement membrane matrix-coated upper chambers in a transwell plate with 8.0-μM pores (Corning Costar, NY, USA). For cell migration assays, cells were seeded into transwell plates with 8.0 μm pore polycarbonate membrane insert (Corning Costar, NY, USA). The lower portion of the chamber contained 30% FBS for use as a chemoattractant. After 24 h (48 h), the number of cells migrating (invading) into the lower chamber were counted using Cell Titer-Glo Assays. Invasive and migration abilities were determined by the number of viable cells in the lower chamber.

Gene Expression Profiling and Kyoto Encyclopedia of Genes and Genomes (KEGG) Pathway Enrichment Analysis

HBL-1 cells were treated with the indicated drug alone or in combination for 24 h, and then total RNA was isolated. Total RNA (3 μg) was converted to cDNA using TransScript First-Strand cDNA Synthesis SuperMix. RNA quantification and qualification, library preparation, clustering and sequencing, read mapping and data processing were performed in Novogene Bioscience (Beijing, China). Differential expression analysis of two groups (two biological replicates per condition) was performed using the DESeq2 R package (1.16.1). Corrected P-value of 0.05 and absolute foldchange of 2 were set as the threshold for significantly differential expression. To analyze the underlying mechanism of the sets of genes which were differentially expressed following each treatment, we used clusterProfiler R package to test the statistical enrichment of differential expression genes in Kyoto Encyclopedia of Genes and Genomes (KEGG) pathways.

Lentivirus Packing and Infection

Lentiviral vectors (GV493) containing green fluorescent protein (GFP) (shControl) or NOTCH1-specific short hairpin RNA (shNOTCH1, sequence #1: 5′-TGCCAACATCCAGGACAACAT-3′ (SEQ ID NO:5)) were constructed, packed, and purified by Genechem (Shanghai, China). Cells were infected with shControl, shNOTCH1, at MOI 1: 100 and cultured for >72 h to be used for the downstream experiments. The depletion efficiency was assessed by western blot analysis.

Detection of Treatment Efficacy in vivo

All animal experiments were performed in compliance with the Guide for the Care and Use of Laboratory Animals and in accordance with the ethical guidelines of CrownBio (Beijing, China). Female immune-deficient NPG mice (NOD -Prkdc^{scid}I12rg^{null}), six to eight weeks old, were obtained from HFK Bioscience Co. Ltd. (Beijing, China). HBL-1 tumor cells (2.5×10⁶) in serum-free medium with matrigel (1:1 ratio) were injected subcutaneously into the area under the right flank of each mouse. When the tumor reached 100-150 mm³, mice were randomly divided into four groups (control, treated with enzastaurin, treated with ibrutinib, treated with both enzastaurin and ibrutinib). Enzastaurin (50 mg/kg, dissolved in 10% Acacia) was administered twice daily orally and/or ibrutinib (12 mg/kg, dissolved in 1% methylcellulose, 0.4% Cremophor® EL) was administered once daily orally for 21 days. Tumor volume (V) and body weight were monitored two to three times per week. The tumor volume (V) was calculated as V=(length×width²)/2. Tumor tissue samples were collected from all groups at 4 h after the last dose.

Statistical Analysis

All experiments in vitro were independently done at least three times. All values are expressed as mean ±SEM. The SPSS 22.0 statistical software (IBM, New York, N.Y., USA) was used for all analyses. Data were analyzed using paired or unpaired Student's t test comparisons or one-way ANOVA. P values<0.05 were accepted as statistically significant. The combination index (CI) for drug combination was determined according to the Chou-Talalay method using the CalcuSyn software (version 2, Biosoft, Cambridge, UK) [26]. CI values<1, =1, and >1 indicate synergistic effects, additive effects, and antagonistic effects, respectively.

Results

Enzastaurin Inhibited Proliferation of ABC and GCB Cell Lines in a Dose-Dependent Manner and Up-Regulated Phosphorylation of BTK

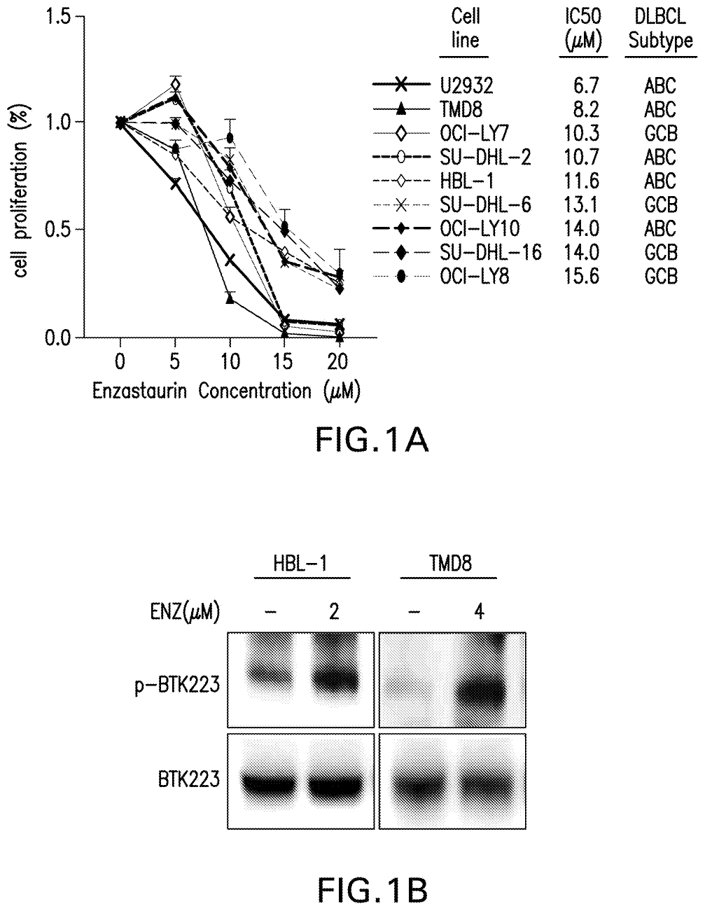

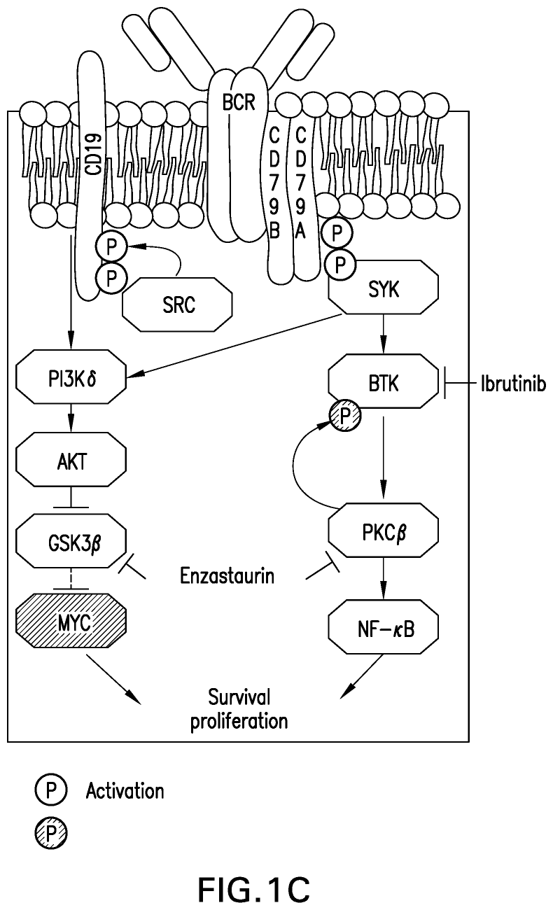

To determine the effect of enzastaurin on the survival of DLBCL cell lines, we cultured 9 cell lines in the presence of enzastaurin (0 to 20.0 μM) for 72 h. The cell lines were cultured with DMSO alone (carrier) or enzastaurin in DMSO. Cell viability was measured by Cell Titer-Glo luminescent cell viability assay. Each cell line was analysed in triplicate, and FIG. 1a shows the average of three replications and standard deviation. As shown in FIG. 1a, treatment with enzastaurin resulted in a dose-dependent inhibition of cell proliferation, with a 50% inhibitory concentration (IC50) between 6.7 and 16 μM (FIG. 1a). These results demonstrated that enzastaurin triggers cytotoxicity in both ABC and GCB types of DLBCL cell lines.

PKCβ is a common downstream target of BTK. Surprisingly, we observed that HBL-1 and TMD8 cells exhibited a significant upregulation of p-BTK upon treatment with enzastaurin (FIG. 1b shows Western blot analysis of p-BTK levels in each cell line after treatment for 2 hours with enzastaurin in DMSO or vehicle alone). Hence, inhibition of PKCβ is therapeutically effective in DLBCL cells, but leads to positive regulation of BCR signalling. Pharmacological inhibition of enzastaurin only blocks some branches of BCR signalling, and inactivation of the pathway can be compensated by others. FIG. 1c shows the BCR pathway, where compensatory pathways may limit enzastaurin's effectiveness in DLBCL, especially as a monotherapy.

Inhibition Rate of DLBCL Cells Independent of the Molecular Subtype or Responsiveness to Ibrutinib.

In order to assess the synergistic effect of enzastaurin and ibrutinib, inhibition of cell growth by enzastaurin alone, ibrutinib alone, or a combination of the two was measured in five cell lines (HBL-1, TMD8, SU-DHL-2, SU-DHL-6 and OCL-LY7) at various concentrations: enzastaurin was tested at 2-12 μM concentrations, and ibrutinib was tested at concentrations of 0.002 to 10 μM, as shown in FIG. 2a, for 72 hours. Cell Titer-Glo luminescent assays were used to detect the rate of inhibition. From the data, combination index (CI) values were calculated (FIG. 2b) using CalcuSSyn software. CI values less than 1.0 denote synergy. The combination treatment showed a strong synergistic inhibitory effect on the growth of HBL-1, TMD8, SU-DHL-6 and OCL-LY7 cell lines at all tested doses, with CI value ranging from 0.239 to 0.686. The synergistic effects in SU-DHL2 were weak at the lower ibrutinib concentrations (CI=0.92), but significant at 10 μM ibrutinib. The combinations of enzastaurin and ibrutinib exhibited synergistic effects in GCB and ABC DLBCL cell lines in nearly all dosage combinations examined (CI<1, FIG. 2b).

Time course analysis of cell death further confirmed that longer time exposure to the combination treatment inhibited cell proliferation more significantly (FIG. 2c). For this experiment, cells were treated with enzastaurin and ibrutinib at the concentrations of enzastaurin (ENZ), Ibrutinib (IB), or both together (Combo) as indicated in FIG. 2c for 24, 48 and 72 hours, and cell viability was monitored by Cell Titer-Glo luminescent assay. Inhibition rates were calculated as (1-treated/vehicle)×100%. The data are presented as the mean value from triplicate observations, plus or minus the standard deviation. Taken together, the combination of enzastaurin and ibrutinib showed long-term synergistic effects on the survival and proliferation of DLBCL cells independent of their subtype.

The Combination of Enzastaurin and Ibrutinib Promoted Apoptosis and Induced G1 Phase Arrest in DLBCL Cells

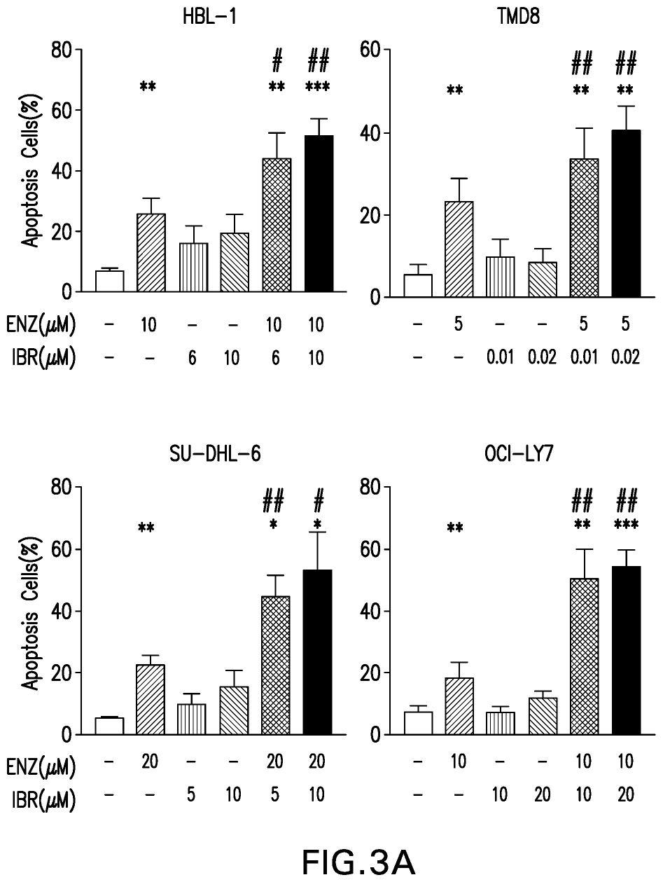

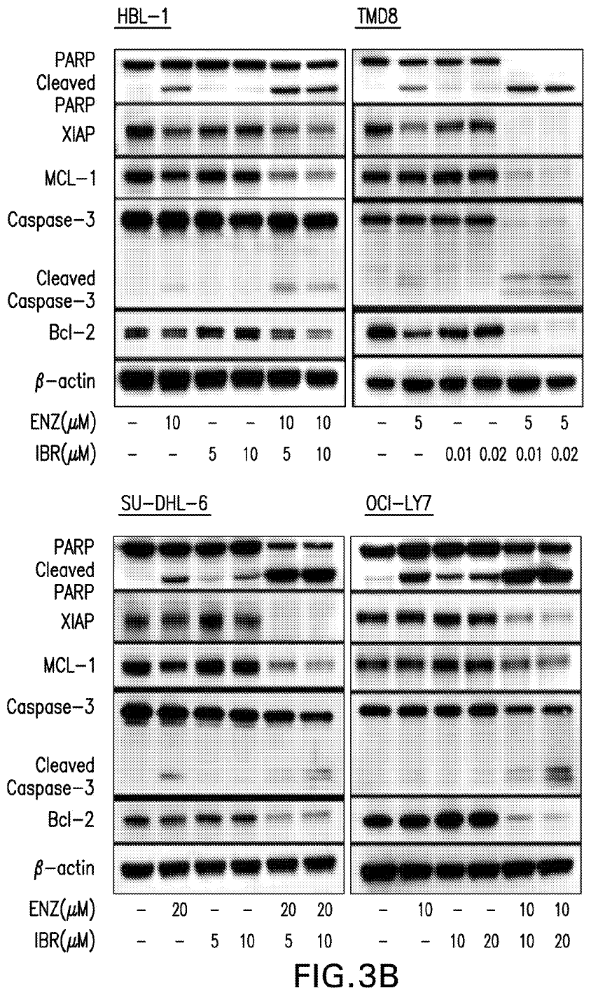

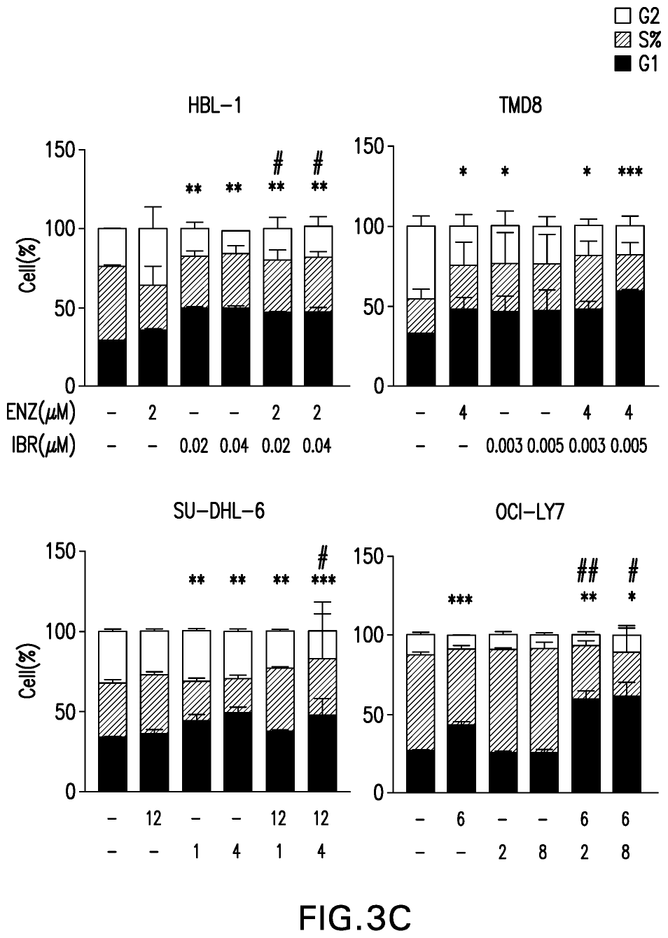

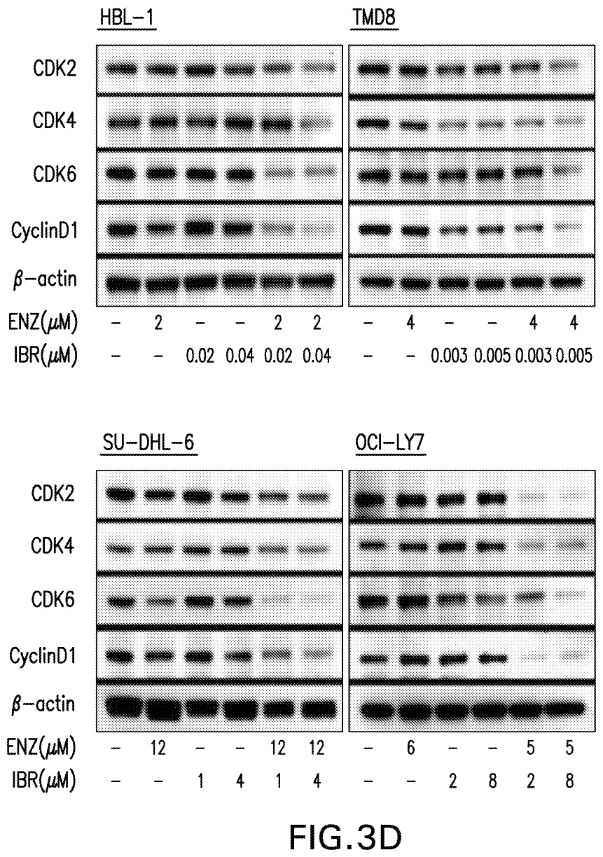

To determine whether the inhibition of cell growth by co-treatment with enzastaurin and ibrutinib was associated with apoptosis and/or cell cycle arrest, apoptosis was monitored in four cells lines after 48 h exposure to enzastaurin and/or ibrutinib with indicated concentrations—see FIG. 3a. Cells were stained with annexin V, and apoptosis was assessed via flow cytometry. Apoptosis was evaluated as APC+cells. In HBL-1, the combination of enzastaurin with two different doses of ibrutinib induced 43.8±8.7% or 51.4±5.9% apoptosis as measured by annexin V staining, more than each single agent alone (enzastaurin=25.5±5.4%, ibrutinib=15.9±6.0% and 19.0±6.7%, FIG. 3a). These results indicated that co-treatment with enzastaurin and ibrutinib resulted in a synergistic effect on promoting apoptosis.

Consistent with the flow cytometry results, proteins associated with apoptosis changed accordingly in HBL-1 cells (FIG. 3b). In this experiment, cells were exposed to enzastaurin and/or ibrutinib as indicated (see FIG. 3c) for 48 hours, then proteins were extracted, and levels of proteins associated with apoptosis (PARP, XIAP, MCL-1, caspase-3, Bcl-2, beta-actin) were More specifically, enzastaurin and ibrutinib were able to increase the cleavage of PARP and caspase-3 slightly, but this effect was significantly enhanced when enzastaurin and ibrutinib were applied together (FIG. 3b). The combination treatment also induced a sharp degradation in the expression of several anti-apoptotic Bcl-2 family members, including Mcl-1, XIAP, and Bcl-2. A similar effect was also observed in TMD8, SU-DHL-6 and OCL-LY7 cells (FIG. 3b). In sum, the above results proved that the combination use of enzastaurin and ibrutinib led to increased apoptosis through caspase-dependent and mitochondrial pathways in DLBCL cells, which finally induced cytotoxicity in DLBCL cells.

The cell cycle histograms further demonstrated the effects of drug combinations on cell cycle (FIG. 3c). For this study, cells were treated with ibrutinib and/or enzastaurin as indicated for 48 hr, then the cells were stained with propidium iodide (PI). Cell cycle was assessed using flow cytometry. HBL-1 cells in G1 phase increased from 28.5±0.05% in the control group to 46.4±0.84% and 47.2±3.12% in combination treatment group, which correlated with a decrease in cells of S phase. Similar results were also obtained for TMD8, SU-DHL-6 and OCL-LY7 cells (FIG. 3c). The graphs represent average values from three replications and the error bars show the standard deviations. Statistically significant results are indicated by * (p<0.05), ** (p<0.01), or *** (p<0.001) above the bars when compared with control group, and with # (p<0.05) or ## (p<0.01) above the bars when compared with the enzastaurin-alone group.

In addition, the level of expression or presence of marker proteins associated with G1/S transition, CDK2, CDK4, CDK6 and Cyclin D1, were assessed in these cells as shown in FIG. 3d. Proteins were extracted after the 48-hour treatment with enzastaurin and/or ibrutinib, and the target proteins were analysed by Western blot. The combination treatments sharply decreased the levels of the marker proteins, while treatment with either single drug had a minor effect on the expression or presence of the proteins associated with G1/S transition. Similar trends can be seen in the other three cell lines (FIG. 3d). These data suggest that the combinations of enzastaurin and ibrutinib induced G1 phase arrest, and co-treatment therapy suppressed cell proliferation resulting in part from cell cycle arrest and in part from apoptotic pathways.

Treatment with Low Doses of Enzastaurin and Ibrutinib Synergistically Inhibits Migration and Invasion in DLBCL

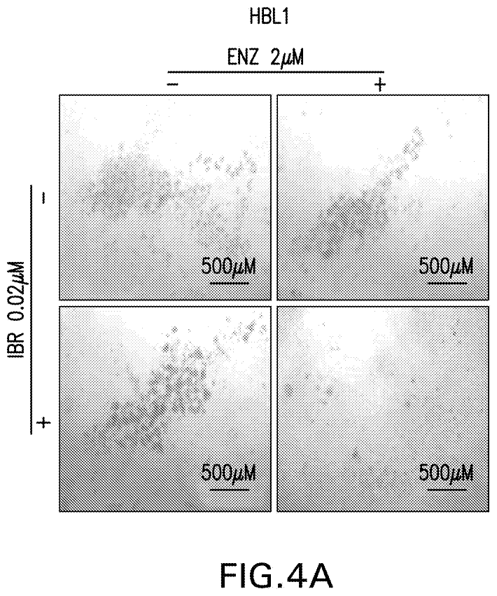

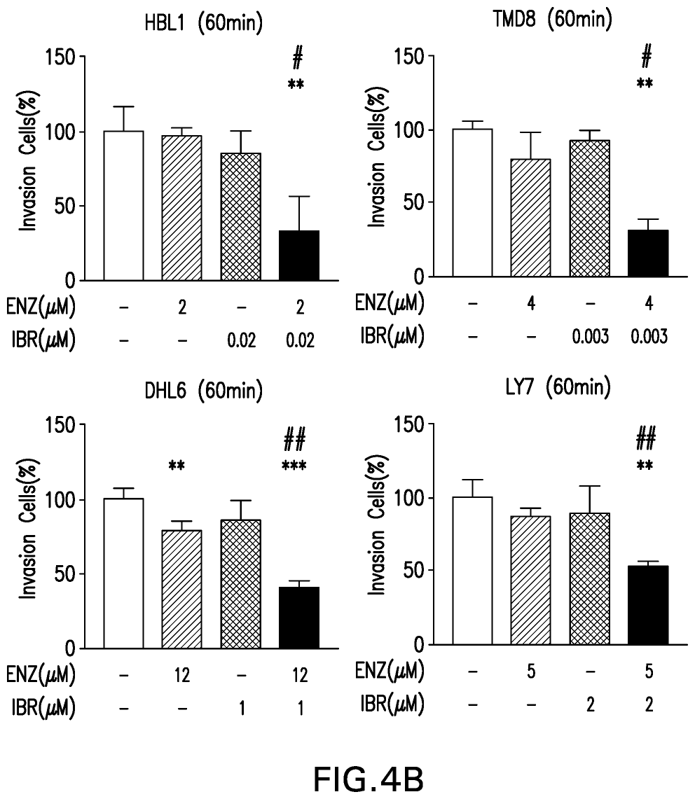

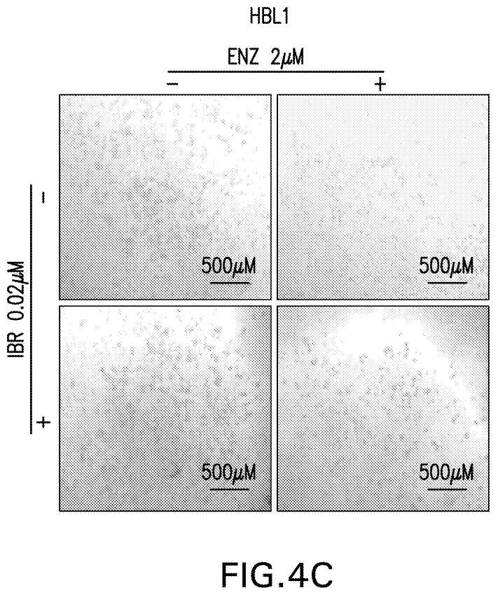

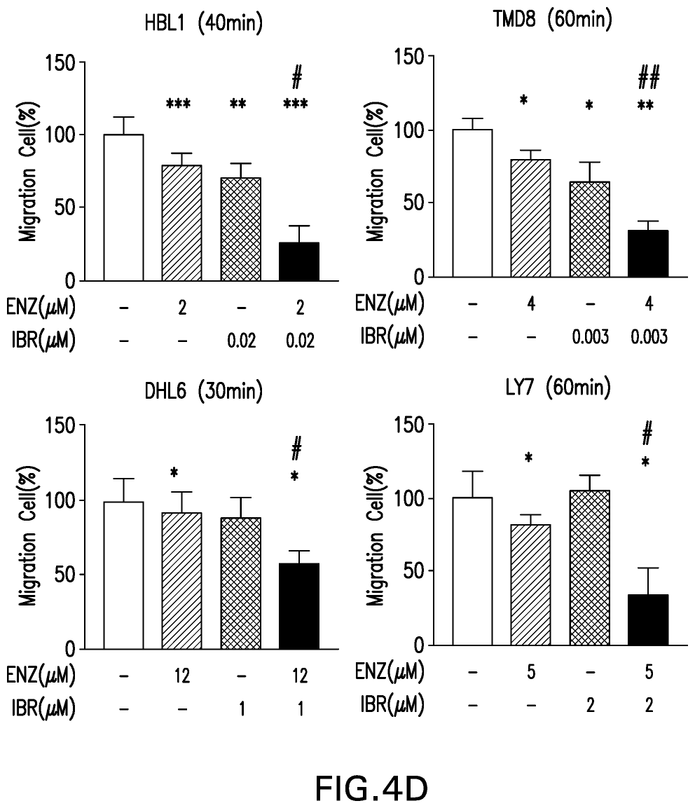

In order to evaluate the possible effects of treatment with low doses of enzastaurin and ibrutinib on cell motility, cell migration and invasion assays were performed using DLBCL cells. HBL-1 cells were pre-treated with 2 micromolar enzastaurin and/or 0.02 micromolar ibrutinib for the indicated time (30-60 min), then placed in a transwell plate of a Corning migration chamber. For the invasion test (not for the migration test), the transwell plate was pre-coated with Matrigel. After 48 hours, the extent of migration or invasion (measured at 24 hours) was assessed, by counting cells in the lower chamber, and expressed as a percentage of controls. For invasive abilities, treatment with enzastaurin or ibrutinib alone slightly reduced invasive abilities to 97.0% and 85.0% of control, respectively, in HBL-1 cells. Invasive abilities were notably suppressed in the combination group (32.8%) treated with enzastaurin and ibrutinib, relative to the control cells (FIG. 4a). In the migration tests, the single agents reduced migration abilities respectively to 79.0% and 70.2% of control in HBL-1 cells. In contrast, the number of cells treated with both enzastaurin and ibrutinib that passed through the membrane was reduced to approximately 25.5% of the control level (FIG. 4b). A similar tendency was found when the experiments were repeated in TMD8, SU-DHL-6 and OCL-LY7 cell lines, as shown in the invasion and migration histograms (FIG. 4b, FIG. 4d). For the histograms in FIGS. 4b and 4d, statistical significance is indicated by * (p<0.05) or ** (p<0.01) or *** (p<0.001) above the bars when compared to control, or by # (p<0.05) or ## (p<0.01) above the bars when compared with the enzastaurin-alone group. These findings show that co-administration of enzastaurin and ibrutinib can work synergistically to reduce cell migration and invasion, which play essential roles in DLBCL cell motility, and are expected to correlate with disease progression.

Combinations of Enzastaurin and Ibrutinib Synergistically Inhibit Downstream Signalling Pathway

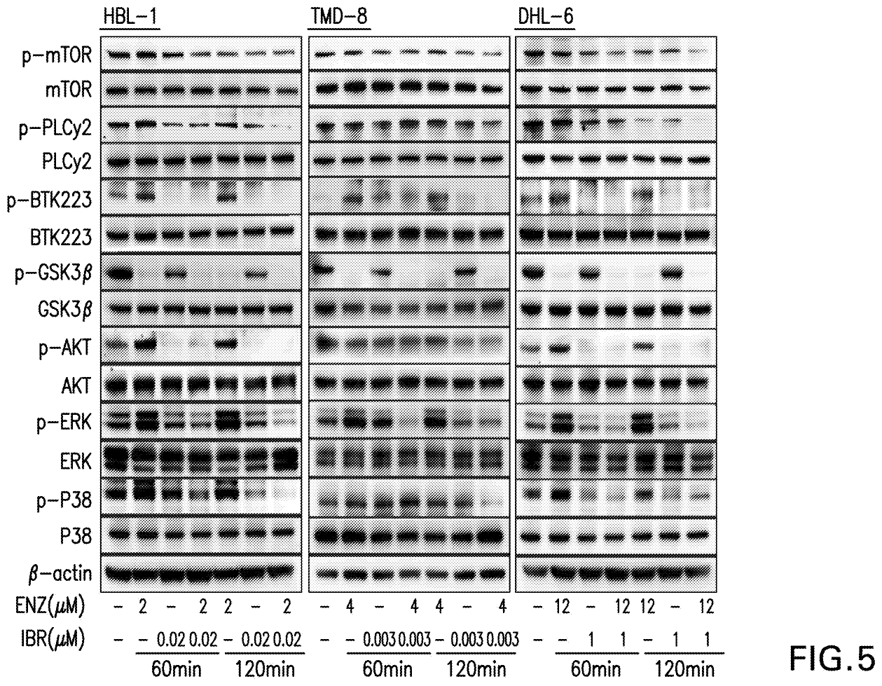

To gain insight into the mechanism underlying the proliferative effect of the combination of enzastaurin and ibrutinib in DLBCL models, the changes in signal transduction pathways caused by each alone and by the combination were examined. HBL-1, TMD-8, and SU-DHL-6 cells were treated with low doses of enzastaurin monotherapy for 60 min and 120 min. as summarized in FIG. 5, and proteins were harvested from the cells for analysis by Western blot. Enzastaurin alone clearly reduced the phosphorylation of glycogen synthase kinase 3β (GSK3β), which serve as a biomarker for enzastaurin activity. Short-term and low-dose enzastaurin treatments do not significantly impact on the PKCβ phosphorylation (data not shown), even increasing p-BTK, p-ERK and p-AKT expression. Similarly, treatment with Ibrutinib alone reduced phosphorylation of BTK and AKT, accompanied by a mild effect on phosphorylation of mTOR, PLCγ2, ERK and P38. However, treatment with both enzastaurin and ibrutinib resulted in a further reduction in phosphorylation of ERK, mTOR, PLCγ2, P38 as compared to each monotherapy alone (FIG. 5). These results were also confirmed in TMD8 and SU-DHL-6 cells. Overall, in contrast to single treatment, the combination of enzastaurin and ibrutinib seems to be more effective in inhibition of signal transduction in both ABC and GCB cell models, which means co-exposure led to a further enhanced suppression of multiple signals downstream of BCR.

Whole-Transcriptome changes in DLBCL are Induced by Low Doses of Enzastaurin Plus Ibrutinib

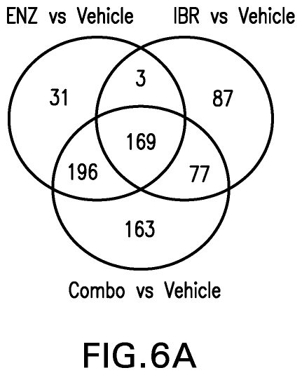

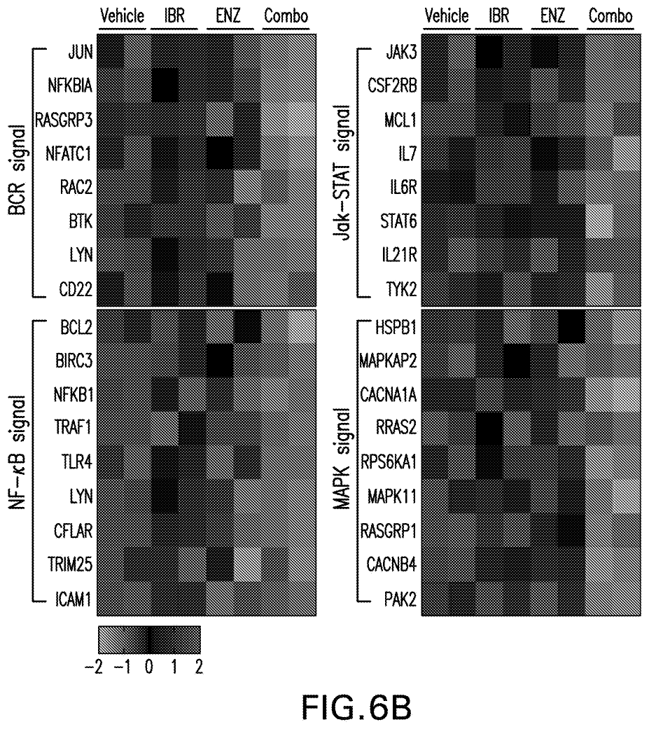

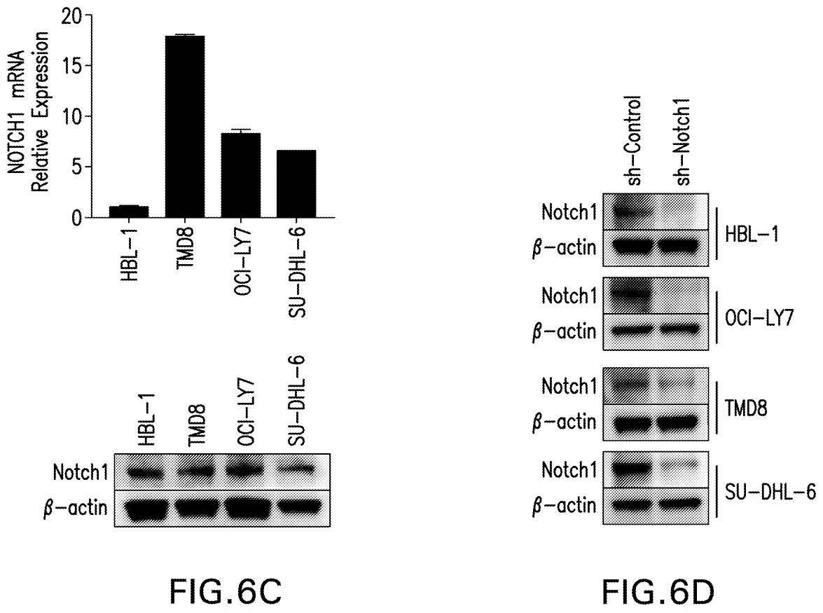

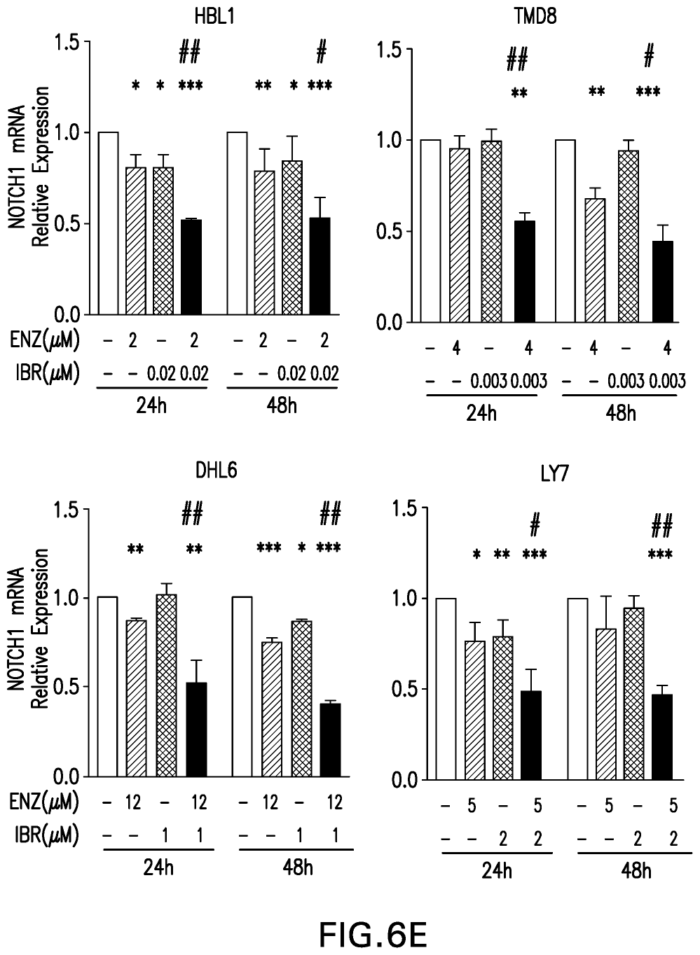

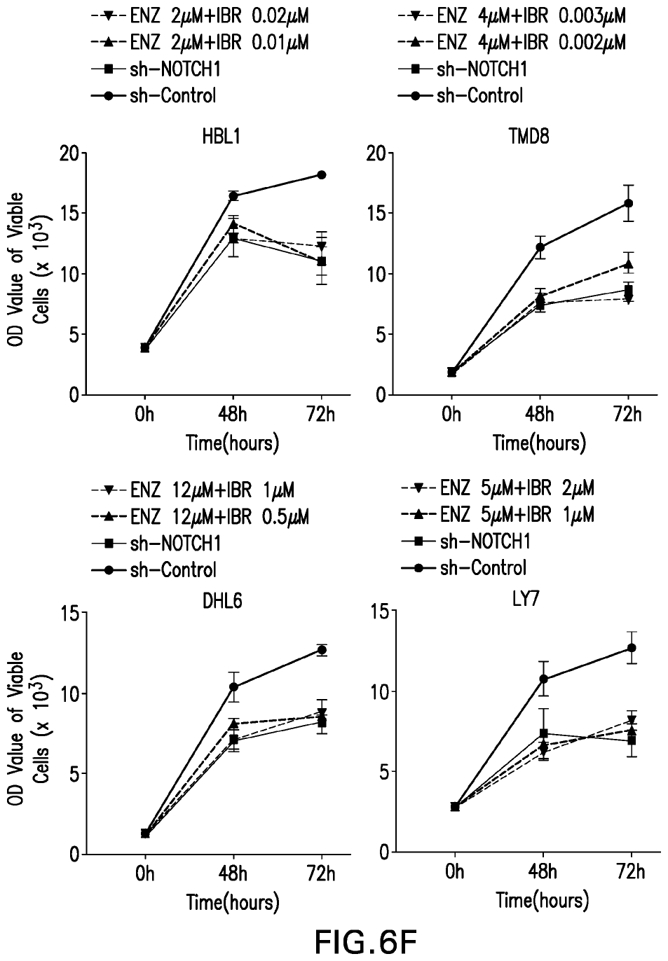

In order to better understand the role of co-treatment with low doses of enzastaurin and ibrutinib in DLBCL cells, we assayed whole-transcriptome changes by RNA-seq using enzastaurin and/or ibrutinib treatments compared to vehicle cells. For this experiment, HBL-1 cells were exposed for 24 hours to 2 micromolar enzastaurin, or 0.02 micromolar ibrutinib, or both together. RNA was collected for sequencing. Several hundred transcripts were identified that were either up- or down-regulated by the various treatments. As the up-regulated genes were not closely associated with these inhibitors, only the down-regulated genes were further studied. The Venn diagram in FIG. 6a illustrates these top down-regulated gene changes produced by different treatments (<2 fold, p<0.05). Enzastaurin and ibrutinib were less efficient than the combination treatment: 339 and 336 transcripts were significantly down, respectively, compared with 605 for the combination treatment. Approximately 91% of transcripts (365 genes) downregulated by enzastaurin and 73% of transcripts (246 genes) downregulated by ibrutinib were contained in the combination treatment group. Additionally, 163 transcripts were also efficiently decreased by the combination treatment, which were not presented in each monotherapy alone (FIG. 6a).

Further analysis identified significantly down-regulated genes from the top ranked pathways (by KEGG). Compared with vehicle treatment control, genes that were significantly down-regulated from top ranked pathways (by KEGG) by the treatments are represented in the heat map (FIG. 6b). Color scale bars represent higher (red) to lower (blue) expression levels, expressed in FPKM values; differences are shown in the colour scale after Z-score transformation. Down-regulated genes were determined by log2foldchange<0. FKPM is fragments per kilobase of exon per million fragments mapped. Co-treatment with low doses of enzastaurin and ibrutinib effectively downregulated genes associated with BCR, NF-κB, JAK and MAPK signalling pathways, and the pathway analysis results were consistent with the immunoblotting analysis (FIGS. 3 and 5). In summary, these results showed that combination of enzastaurin and ibrutinib acted synergistically to regulate whole-transcriptome changes.

To further assess the synergistic anti-tumor effects of enzastaurin and ibrutinib, we detected the mRNA expression of transcripts changed in combination treatment using qRT-PCR. In particular, the expressions of NOTCH1 in DLBCL cell lines were detected using qRT-PCR and Western blot. NOTCH1 knockdown by shRNA was validated by Western blot in HBL-1, TMD9, OCI-LY7, and SU-DHL-6 cells. Beta-actin is used as a loading control. Expressions of NOTCH1 were further confirmed by qRT-PCR. See FIG. 6c, FIG. 6d.

Compared with enzastaurin and ibrutinib monotherapy, combination treatment was able to reduce the mRNA expression of NOTCH1 more significantly (FIG. 6e). A strong body of evidence reveals the important oncogenic role of NOTCH1 in promoting cell metabolism, growth and proliferation, as well as in enhancing the activity of signalling pathways [27-30]. The expression of NOTCH1 mRNA and protein in DLBCL cells were a medium to high level (FIG. 6c). Aberrant NOTCH1 activity has emerged as an important oncogenic regulator of haematological malignancy [30, 31]. The combination effect of enzastaurin and ibrutinib in inhibiting proliferation of DLBCL cells is likely achieved through suppression of NOTCH1 expression.

To validate the role of NOTCH1 in DLBCL cell survival and proliferation, shRNA transfection was used to knock-down NOTCH1 (FIG. 6d). DLBCL cells were transfected with shRNA targeting NOTCH1 or treated with enzastaurin and ibrutinib for 48 hours and 72 hours. Cell viability was then determined using the Cell Titer-Glo luminescent cell viability assay. Results shown in FIG. 6e represent mean and standard deviation from three independent replications; statistical significance is indicated by * (p<0.05) or ** (p<0.01) or *** (p<0.001) above the bars when compared to control, or by # (p<0.05) or ## (p<0.01) above the bars when compared with the enzastaurin-alone group.

Silencing NOTCH1 in DLBCL cells caused anti-proliferative activity towards tumor cells, indicating NOTCH1 expression is key for the survival of DLBCL cells. Surprisingly, a similar proliferation inhibitory effect was detected and consistent with time in NOTCH1 shRNA and co-treatment therapy, indicating that combination treatment might also lead to synergistic anti-DLBCL effects through downregulating NOTCH1 expression (FIG. 6f).

Synergistic Antitumor effects of Enzastaurin and Ibrutinib in DLBCL-Cell-Derived Xenografts.

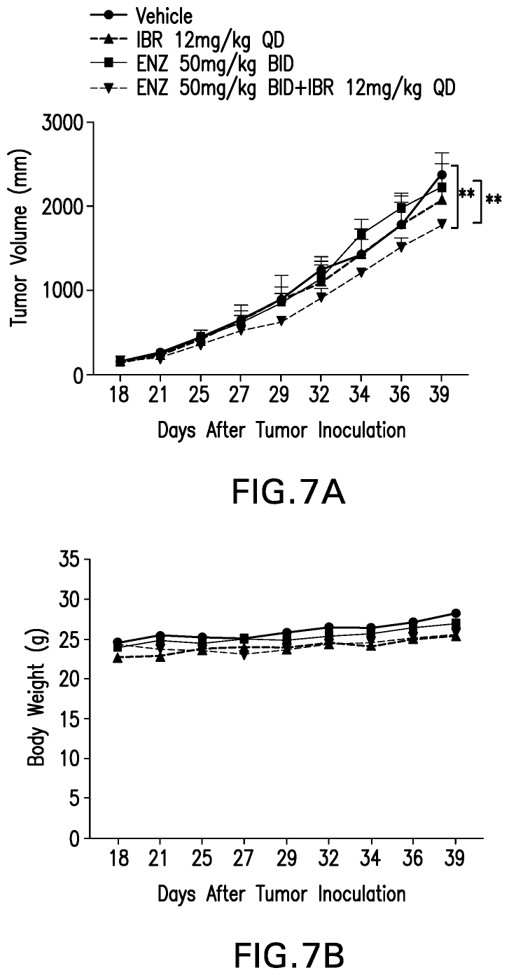

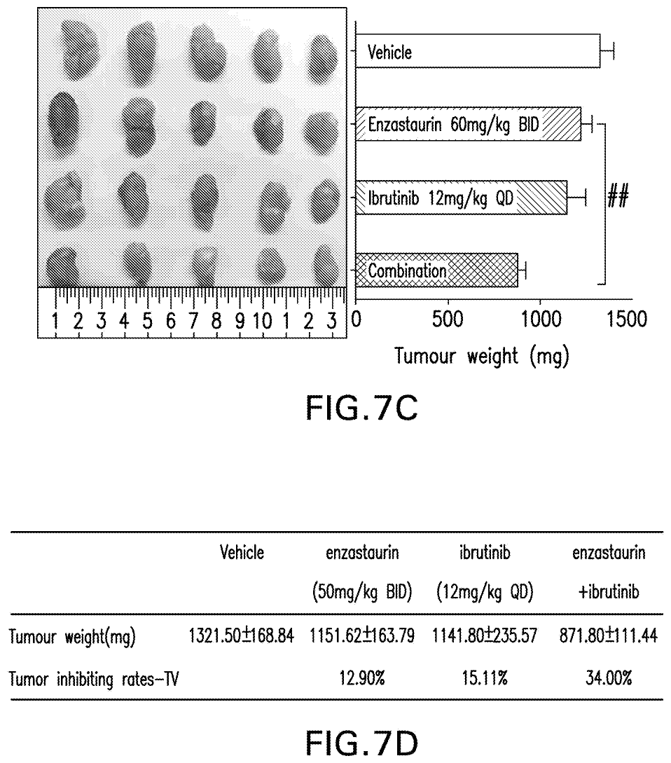

Finally, we assessed the ability of enzastaurin, alone and in combination with ibrutinib, to reduce tumor growth in a lymphoma xenograft model, in which ABC-DLBCL HBL-1 cells were engrafted in NPG mice (FIG. 7). NPG mice were injected subcutaneously with HBL-1 cells (2.5×10⁶ cells) and were randomized into four groups (n=5 per group). The groups were treated, respectively, with enzastaurin, ibrutinib, a combination of enzastaurin and ibrutinib, and vehicle. Enzastaurin was administered orally at a dosage of 50 mg/kg, BID. Ibrutinib was administered orally at a dosage of 12 mg/kg, QD (total daily dosing was 100 mg/kg Enzastaurin per day and 12 mg/kg ibrutinib per day). Tumor size and weight are described as mean +/− standard deviation; statistical significance is indicated by * (p<0.05) or ** (p<0.01) or *** (p<0.001) when compared to control, or by # (p<0.05) or ## (p<0.01) when compared with the enzastaurin-alone group.

Dosing enzastaurin or ibrutinib as monotherapy at the above dosages produced weak activity in reduction of tumor volume, and neither therapeutic agent alone gave a statistically significant reduction of tumor growth. Compared with control and monotherapy, tumor volumes were significantly smaller in mice treated with the combination treatment (p<0.01, FIG. 7a) at 2-3 weeks after treatment was initiated on Day 18 post-inoculation. All treatments were very well tolerated with no significant body weight change in any treatment group (FIG. 7b).

At the end of the experiment (day 39), neither enzastaurin alone (1151.62±163.79 mg) nor ibrutinib alone (1141.80±235.57 mg) produced significant inhibition of tumour growth compared with that of the vehicle group (1321.50±168.84 mg), while the combination robustly suppressed tumour growth and restrained tumour weight (871.80±111.44 mg, FIG. 7c, 7d). In sum, these results demonstrate the synergistic activity using enzastaurin in combination with ibrutinib in preclinical models, which confirms the in vitro findings above. Thus, the combination of enzastaurin and ibrutinib produced a lasting synergistic effect on the survival and proliferation of ABC (HBL-1, TMD8, SU-DHL-2) and GCB (SU-DHL-6, OCI-LY7) DLBCL cell lines.

Biomarker for Patient Selection

Denovo conducted an analysis of enzastaurin's DLBCL clinical data and identified a subset of patients who showed improved survival. Using its proprietary biomarker discovery platform, the company identified a novel biomarker, which was named Denovo Genomic Marker 1 (DGM1). DGM1 and its use as a biomarker for selecting subjects for treatment with enzastaurin are disclosed and described in published patent application WO2018/045240. Data showed that DGM1-positive patients exhibited significantly improved survival over DGM1-negative patients in DLBCL trials of enzastaurin. Enzastaurin is now in a Phase III clinical trial in which DGM1 expression or presence is used to select DLBCL patients who are likely to respond to treatment.

The ENGINE trial (NCT03263026) is assessing enzastaurin in combination with the R-CHOP regimen (which consists of rituximab, cyclophosphamide, doxorubicin, vincristine, and prednisone), against R-CHOP alone, in first-line DLBCL patients, with or without the DGM1 biomarker. The primary endpoint of the trial is overall survival in patients who possess the biomarker, and it has a primary completion date of October 2020.

### Example 2

Material and Methods

Cell Lines and Cell Culture

Diffuse large B cell lymphoma (DLBCL) cell lines, SU-DHL-5 and SU-DHL-6 were purchased from the American Type Culture Collection (ATCC, Manassas, Va.). Cells were grown in RPMI1640 medium (Gibco, Life Technologies, CA, USA) supplemented with 10% fetal bovine serum (Gibco, Life Technology, CA, USA). All cell lines were maintained in a humidified 5% CO2 incubator at 37° C.

Drugs and Reagents

Enzastaurin (LY317615), Acalabrutinib (ACP-196), Spebrutinib (CC-292,AVL-292), and ARQ531 were purchased from Selleckchem (Houston, Tex., USA), Zanubrutinib (BGB-3111) from MedKoo BioSciences (Morrisville, N.C., USA), and Vecabrutinib (SNS-062) form MedChemExpress (Monmouth Junction, N.J., USA). All compounds were initially dissolved in 100% dimethylsulfoxide (DMSO, Sigma Chemical) at a concentration of 10 mM and aliquoted and stored at −20° C.

Cell Proliferation Assay

Cells were seeded in triplicates in 96-well cell culture plates at a density of 2.5×10⁴ per well and treated with Enzastaurin or Bruton tyrosine kinase inhibitor (BTKi) alone, or in combination, at different concentrations for 72 h. After treatment, cell viability was assessed using Cell Titer AQueous One Solution Cell Proliferation Assay Kits (Promega, Madison, Wis., USA). The cell cytotoxicity was measured by BioTek Elx800 microplate reader at absorbance 490 nm. The IC50 values of each drug were calculated from curves of drug concentration 0.014 μM to 10 μM.

Statistical Analysis

All experiments were independently done at least twice. All values are expressed as mean±SEM. Data were analyzed using Student's t test for P values. P values<0.05 were accepted as statistically significant. Compound inhibition effects were calculated by comparison of untreated control cells. The combination index (CI) for drug combination was determined according to the Chou-Talalay method using the CalcuSyn software (version 2, Biosoft, Cambridge, UK). CI values<1, =1, and >1 indicate synergistic effects, additive effects, and antagonistic effects, respectively.

Results

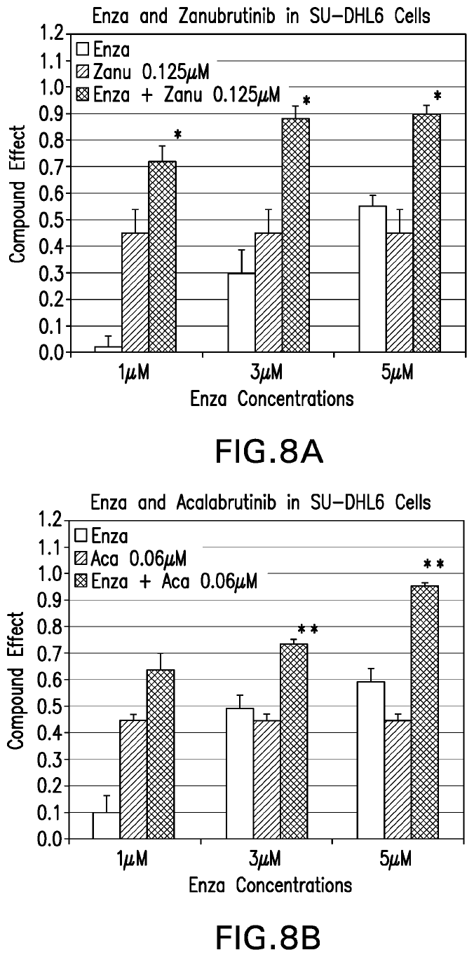

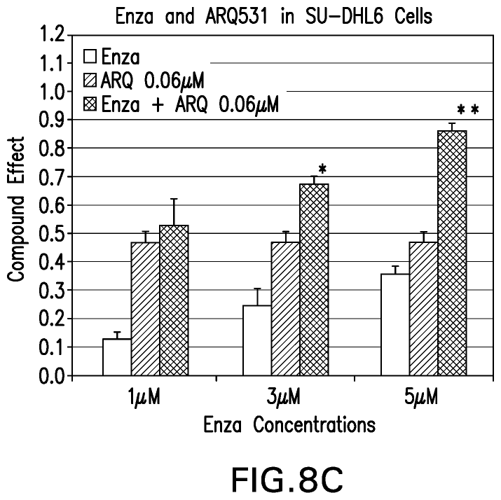

Drug combination is widely used in treatments of many diseases, such as cancers. To determine the combination effects of Enzastaurin and BTK inhibitors on DLBCL cell proliferation, the dose response curves of each drug were determined by treatment of the drug at 0.014 μM-10 μM. The efficacy and 50% inhibitory concentrations (IC50) were calculated and used for combination dose selection. Treatments of enzastaurin resulted in a dose-dependent inhibition of cell proliferation with the IC50 at 3.6 μM in SU-DHL-5, and 5.9 μM in SU-DHL-6 cell lines (data not shown). For combination study, Enzastaurin 1 μM, 3 μM, and 5 μM were selected for treatment combinations with BTK inhibitors, Zanubrutinib, Acalabrutinib, and ARQ531 at different concentrations. As shown in FIG. 8, treatments of enzastaurin at 3 different doses combined with zanubrutinib at 0.125 μM showed significant antiproliferation activities of SU-DHL-6 cells, while enzastaurin at 3 similar doses combined with acalabrutinib and ARQ531 achieved similar effects at lower concentrations of 0.06 μM.

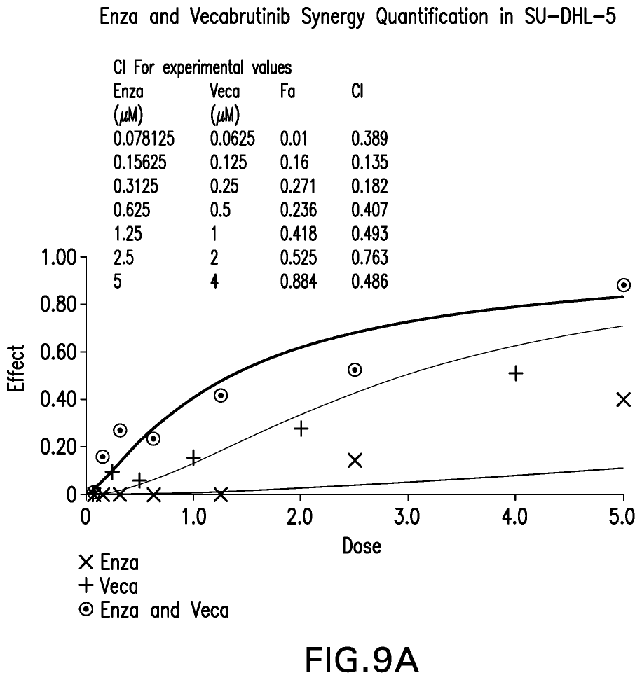

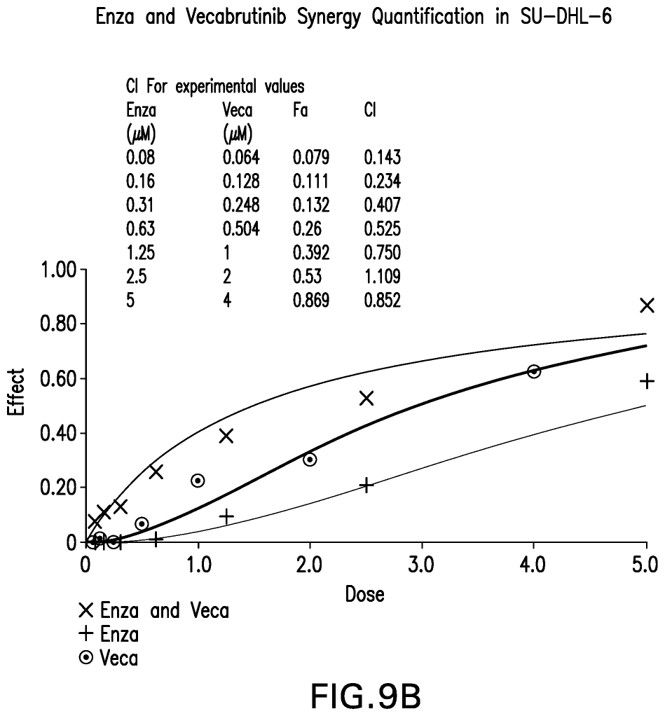

For Enzastaurin synergy quantification study, the constant ratio concentration method developed by Chou-Talalay was used for the combination experiment design. The CalcuSyn software was practiced for data analysis and combination index (CI) calculation. The resulting CI offers quantitative definition for additive effect (CI=1), synergism (CI<1), and antagonism (CI>1) in drug combinations. Enzastaurin and BTK inhibitor Vecabrutinib were investigated in this assay. Enzastaurin started at a concentration of 5 μM alone or in combination of Vecabrutinib 4 μM, the constant ratio (IC50 ratio), and both drugs were diluted at 1:1 dilution to final 0.08 μM and 0.06 μM respectively followed the same IC50 ratio. As shown in FIG. 9, the combination of Enzastaurin and Vecabrutinib resulted in the synergistic cell viability decreasing than single drugs after treatments for 72 h in both cell lines. In SU-DHL-5 cells, the combination treatment showed strong synergistic inhibitory effect on the cell proliferation with CI<1 for all selected doses from Enzastaurin 0.08-5 μM (CI from 0.135-0.78) and 0.08-0.6 μM in SU-DHL-6 cells (CI from 0.143-0.852).

The detailed description set-forth above is provided to aid those skilled in the art in practicing the present invention. However, the invention described and claimed herein is not to be limited in scope by the specific embodiments herein disclosed: the examples and embodiments are intended as illustration of several aspects of the invention. Any equivalent embodiments are intended to be within the scope of this invention. Indeed, various modifications of the invention in addition to those shown and described herein will become apparent to those skilled in the art from the foregoing description which do not depart from the spirit or scope of the present inventive discovery. Such modifications are also intended to fall within the scope of the appended claims.

All publications, patents, patent applications and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present invention.

Certain cited references are listed below.

1. A clinical evaluation of the International Lymphoma Study Group classification of non-Hodgkin's lymphoma. The Non-Hodgkin's Lymphoma Classification Project. Blood, 1997. 89(11): p. 3909-18.

2. Alizadeh, A. A., et al., Distinct types of diffuse large B-cell lymphoma identified by gene expression profiling. Nature, 2000. 403(6769): p. 503-11.

3. Wilson, W. H., et al., Targeting B cell receptor signaling with ibrutinib in diffuse large B cell lymphoma. Nat Med, 2015. 21(8): p. 922-6.

4. Roschewski, M., L. M. Staudt, and W. H. Wilson, Diffuse large B-cell lymphoma-treatment approaches in the molecular era. Nat Rev Clin Oncol, 2014. 11(1): p. 12-23.

5. Young, R. M. and L. M. Staudt, Targeting pathological B cell receptor signalling in lymphoid malignancies. Nat Rev Drug Discov, 2013. 12(3): p. 229-43.

6. Duhren-von Minden, M., et al., Chronic lymphocytic leukaemia is driven by antigen-independent cell-autonomous signalling. Nature, 2012. 489(7415): p. 309-12.

7. Paul, J., et al., Simultaneous Inhibition of PI3Kdelta and PI3Kalpha Induces ABC-DLBCL Regression by Blocking BCR-Dependent and -Independent Activation of NF-kappaB and AKT. Cancer Cell, 2017. 31(1): p. 64-78.

8. Davis, R. E., et al., Chronic active B-cell-receptor signalling in diffuse large B-cell lymphoma. Nature, 2010. 463(7277): p. 88-92.

9. Phelan, J. D., et al., A multiprotein supercomplex controlling oncogenic signalling in lymphoma. Nature, 2018.

10. Pfeifer, M., et al., PTEN loss defines a PI3K/AKT pathway-dependent germinal center subtype of diffuse large B-cell lymphoma. Proc Natl Acad Sci USA, 2013. 110(30): p. 12420-5.

11. Erdmann, T., et al., Sensitivity to PI3K and AKT inhibitors is mediated by divergent molecular mechanisms in subtypes of DLBCL. Blood, 2017. 130(3): p. 310-322.

12. Gaudio, E., et al., Combination of the MEK inhibitor pimasertib with BTK or PI3K-delta inhibitors is active in preclinical models of aggressive lymphomas. Ann Oncol, 2016. 27(6): p. 1123-8.

13. T. Bourhill*, A. Narendran2, R. N. Johnston1, Enzastaurin: A lesson in drug development.pdf. Critical Reviews in Oncology/Hematology, 2017.

14. Cosenza, M., et al., The combination of bortezomib with enzastaurin or lenalidomide enhances cytotoxicity in follicular and mantle cell lymphoma cell lines. Hematol Oncol, 2015. 33(4): p. 166-75.

15. Civallero, M., et al., Genomic profiling of enzastaurin-treated B cell lymphoma RL cells. Hematol Oncol, 2011. 29(3): p. 154-6.

16. Bodo, J., et al., HDAC inhibitors potentiate the apoptotic effect of enzastaurin in lymphoma cells. Apoptosis, 2011. 16(9): p. 914-23.

17. Crump, M., et al., Randomized, Double-Blind, Phase III Trial of Enzastaurin Versus Placebo in Patients Achieving Remission After First-Line Therapy for High-Risk Diffuse Large B-Cell Lymphoma. J Clin Oncol, 2016. 34(21): p. 2484-92.

18. Robertson, M. J., et al., Phase II study of enzastaurin, a protein kinase C beta inhibitor, in patients with relapsed or refractory diffuse large B-cell lymphoma. J Clin Oncol, 2007. 25(13): p. 1741-6.

19. Carducci, M. A., et al., Phase I dose escalation and pharmacokinetic study of enzastaurin, an oral protein kinase C beta inhibitor, in patients with advanced cancer. J Clin Oncol, 2006. 24(25): p. 4092-9.

20. Wang, M. L., et al., Targeting BTK with ibrutinib in relapsed or refractory mantle-cell lymphoma. N Engl J Med, 2013. 369(6): p. 507-16.

21. Byrd, J. C., et al., Targeting BTK with ibrutinib in relapsed chronic lymphocytic leukemia. N Engl J Med, 2013. 369(1): p. 32-42.

22. Advani, R. H., et al., Bruton tyrosine kinase inhibitor ibrutinib (PCI-32765) has significant activity in patients with relapsed/refractory B-cell malignancies. J Clin Oncol, 2013. 31(1): p. 88-94.

23. Tarafdar, A. and A. M. Michie, Protein kinase C in cellular transformation: a valid target for therapy? Biochem Soc Trans, 2014. 42(6): p. 1556-62.

24. Kang, S. W., et al., PKCbeta modulates antigen receptor signaling via regulation of Btk membrane localization. Embo j, 2001. 20(20): p. 5692-702.

25. Li, J., et al., The mTOR kinase inhibitor everolimus synergistically enhances the anti-tumor effect of the Bruton's tyrosine kinase (BTK) inhibitor PLS-123 on Mantle cell lymphoma. Int J Cancer, 2018. 142(1): p. 202-213.

26. Chou, T. C., Drug combination studies and their synergy quantification using the Chou-Talalay method. Cancer Res, 2010. 70(2): p. 440-6.

27. Jundt, F., et al., Activated Notch1 signaling promotes tumor cell proliferation and survival in Hodgkin and anaplastic large cell lymphoma. Blood, 2012. 99(9): p. 3398-403.

28. Hales, E. C., J. W. Taub, and L. H. Matherly, New insights into Notch1 regulation of the PI3K-AKT-mTOR1 signaling axis: targeted therapy of gamma-secretase inhibitor resistant T-cell acute lymphoblastic leukemia. Cell Signal, 2014. 26(1): p. 149-61.

29. Bi, P. and S. Kuang, Notch signaling as a novel regulator of metabolism. Trends Endocrinol Metab, 2015. 26(5): p. 248-55.

30. Sanchez-Martin, M. and A. Ferrando, The NOTCH1-MYC highway toward T-cell acute lymphoblastic leukemia. Blood, 2017. 129(9): p. 1124-1133.

31. Paganin, M. and A. Ferrando, Molecular pathogenesis and targeted therapies for NOTCH1-induced T-cell acute lymphoblastic leukemia. Blood Rev, 2011. 25(2): p. 83-90.

32. Sehn, L. H., et al., Introduction of combined CHOP plus rituximab therapy dramatically improved outcome of diffuse large B-cell lymphoma in British Columbia. J Clin Oncol, 2005. 23(22): p. 5027-33.

33. Ngo, V. N., et al., Oncogenically active MYD88 mutations in human lymphoma. Nature, 2011. 470(7332): p. 115-9.

34. Lenz, G., et al., Oncogenic CARD11 mutations in human diffuse large B cell lymphoma. Science, 2008. 319(5870): p. 1676-9.

35. Jerkeman, M., et al., Targeting of B-cell receptor signalling in B-cell malignancies. J Intern Med, 2017. 282(5): p. 415-428.

36. Neri, A., et al., The oral protein-kinase C beta inhibitor enzastaurin (LY317615) suppresses signalling through the AKT pathway, inhibits proliferation and induces apoptosis in multiple myeloma cell lines. Leuk Lymphoma, 2008. 49(7): p. 1374-83.

37. Baumann, P., et al., Inhibitors of protein kinase C sensitise multiple myeloma cells to common genotoxic drugs. Eur J Haematol, 2008. 80(1): p. 37-45.

38. Moreau, A. S., et al., Protein kinase C inhibitor enzastaurin induces in vitro and in vivo antitumor activity in Waldenstrom macroglobulinemia. Blood, 2007. 109(11): p. 4964-72.

39. Querfeld, C., et al., The selective protein kinase C beta inhibitor enzastaurin induces apoptosis in cutaneous T-cell lymphoma cell lines through the AKT pathway. J Invest Dermatol, 2006. 126(7): p. 1641-7.

40. Shipp, M. A., et al., Diffuse large B-cell lymphoma outcome prediction by gene-expression profiling and supervised machine learning. Nat Med, 2002. 8(1): p. 68-74.

41. Hans, C. P., et al., Expression of PKC-beta or cyclin D2 predicts for inferior survival in diffuse large B-cell lymphoma. Mod Pathol, 2005. 18(10): p. 1377-84.

42. Cosenza, M., et al., Combination of low doses of enzastaurin and lenalidomide has synergistic activity in B-non-Hodgkin lymphoma cell lines. Ann Hematol, 2012. 91(10): p. 1613-22.

43. Civallero, M., et al., NVP-BEZ235 alone and in combination in mantle cell lymphoma: an effective therapeutic strategy. Expert Opin Investig Drugs, 2012. 21(11): p. 1597-606.

44. Woyach, J. A., et al., BTK(C481S)-Mediated Resistance to Ibrutinib in Chronic Lymphocytic Leukemia. J Clin Oncol, 2017. 35(13): p. 1437-1443.

45. Battistello, E., et al., Pan-SRC kinase inhibition blocks B-cell receptor oncogenic signaling in non-Hodgkin lymphoma. Blood, 2018. 131(21): p. 2345-2356.

46. Mathews Griner, L. A., et al., High-throughput combinatorial screening identifies drugs that cooperate with ibrutinib to kill activated B-cell-like diffuse large B-cell lymphoma cells. Proc Natl Acad Sci USA, 2014. 111(6): p. 2349-54.

47. Dasmahapatra, G., et al., The Bruton tyrosine kinase (BTK) inhibitor PCI-32765 synergistically increases proteasome inhibitor activity in diffuse large-B cell lymphoma (DLBCL) and mantle cell lymphoma (MCL) cells sensitive or resistant to bortezomib. Br J Haematol, 2013. 161(1): p. 43-56.

48. Yang, Y., et al., Exploiting synthetic lethality for the therapy of ABC diffuse large B cell lymphoma. Cancer Cell, 2012. 21(6): p. 723-37.

49. Lee, C. S., M. A. Rattu, and S. S. Kim, A review of a novel, Bruton's tyrosine kinase inhibitor, ibrutinib. J Oncol Pharm Pract, 2016. 22(1): p. 92-104.

50. Schmitz, R., et al., Genetics and Pathogenesis of Diffuse Large B-Cell Lymphoma. N Engl J Med, 2018. 378(15): p. 1396-1407.

51. Karube, K., et al., Integrating genomic alterations in diffuse large B-cell lymphoma identifies new relevant pathways and potential therapeutic targets. Leukemia, 2018. 32(3): p. 675-684.

52. Reddy, A., et al., Genetic and Functional Drivers of Diffuse Large B Cell Lymphoma. Cell, 2017. 171(2): p. 481-494.e15.

1. A composition comprising enzastaurin, or a pharmaceutically acceptable salt thereof, and an inhibitor of BTK.

2. The composition of claim 1, wherein the inhibitor of BTK is selected from M7583, ibrutinib, acalabrutinib, zanubrutinib, CT-1530, DTRMWXHS-12, spebrutinib besylate, vecabrutinib, evobrutinib, tirabrutinib, fenebrutinib, poseltinib, BMS-986142, ARQ-531, LOU-064, PRN-1008, ABBV-599, AC-058, ARQ-531, BIM-068, BMS-986195, HWH-486, PRN-2246, TAK-020, GDC-0834, BMX-IN-1, RN486, SNS-062, LFM-A13, PCI-32765 (racemate of ibrutinib), CGI-1746, ONO-4059, and SHR-1459, and their pharmaceutically acceptable salts, and preferably the inhibitor of BTK is selected from M7583, ibrutinib, acalabrutinib, zanubrutinib, CT-1530, DTRMWXHS-12, spebrutinib besylate, vecabrutinib, ARQ-531, and SHR-1459, and their pharmaceutically acceptable salts.

3. The composition of claim 1, wherein the inhibitor of BTK is selected from M7583, Ibrutinib, and Acalabrutinib and the pharmaceutically acceptable salts thereof, and is preferably ibrutinib or a pharmaceutically acceptable salt thereof.

4. (canceled)

5. A therapeutic combination, comprising enzastaurin, or a pharmaceutically acceptable salt thereof, and an inhibitor of BTK.

6. The therapeutic combination of claim 5, wherein the inhibitor of BTK is selected from M7583, ibrutinib, acalabrutinib, zanubrutinib, CT-1530, DTRMWXHS-12, spebrutinib besylate, vecabrutinib, evobrutinib, tirabrutinib, fenebrutinib, poseltinib, BMS-986142, ARQ-531, LOU-064, PRN-1008, ABBV-599, AC-058, ARQ-531, BIIB-068, BMS-986195, HWH-486, PRN-2246, TAK-020, GDC-0834, BMX-IN-1, RN486, SNS-062, LFM-A13, PCI-32765 (racemate of ibrutinib), CGI-1746, ONO-4059, and SHR-1459, and their pharmaceutically acceptable salts, and preferably the BTK inhibitor is selected from M7583, ibrutinib, acalabrutinib, zanubrutinib, CT-1530, DTRMWXHS-12, spebrutinib besylate, vecabrutinib, ARQ-531, and SHR-1459, and the pharmaceutically acceptable salts thereof.

7. (canceled)

8. The therapeutic combination of claim 5, wherein enzastaurin and the inhibitor of BTK are prepared for simultaneous administration or for separate administration.

9. (canceled)

10. An in vivo therapeutic combination, which comprises enzastaurin and a BTK inhibitor in the blood or plasma of a subject.

11. A method to treat or prevent a disorder or disease selected from oncologic conditions, immunological disorders, gastrointestinal disorders, CNS disorders, dermatological disorders, hematological disorders, and metabolic disorders, wherein the method comprises administering to a subject in need of such treatment enzastaurin and an inhibitor of BTK.

12. The method of claim 11, which is a method for treatment or prevention of a cancer selected from chronic lymphocytic leukemia (CLL), extranodal marginal zone B-cell lymphoma, mucosa-associated lymphoid tissue lymphoma (MALT-lymphoma), Waldenstrom's macroglobulinemia, mantle cell lymphoma, relapsed CLL, refractory CLL, follicular lymphoma, adenocarcinoma, metastatic adenocarcinoma (e.g., pancreatic), non-Hodgkin lymphoma, pancreatic cancer, acute lymphocytic leukemia, acute lymphoblastic leukemia, hairy cell leukemia, metastatic breast cancer, acute myelocytic leukemia, acute myeloblastic leukemia, multiple meloma, refractory multiple myeloma, relapsed multiple myeloma, gastric cancer, colorectal cancer, bladder cancer, Hodgkin lymphoma (B-cell Hodgkin lymphoma), metastic melanoma, non-small cell lung cancer, primary CNS lymphoma, renal cell carcinoma, secondary CNS lymphoma, transitional cell carcinoma, urothelial cell carcinoma, nodal marginal B-cell lymphoma, splenic marginal zone B-cell lymphoma, T-cell lymphomas, epithelial ovarian cancer, fallopian tube cancer, peritoneal cancer, (recurrent) head and neck cancer, squamous cell carcinoma, (recurrent) glioblastoma multiforme (GBM), and B-Cell lymphoma including diffuse large B-cell lymphoma.

13. (canceled)

14. A method for treatment or prevention of lymphoma, or for reduction of risk of metastasis or relapse in a subject having been treated for lymphoma, wherein the method comprises administering to a subject in need thereof, enzastaurin, or a pharmaceutically acceptable salt thereof, and an inhibitor of BTK.

15. The method of claim 14, wherein the inhibitor of BTK is selected from M7583, ibrutinib, acalabrutinib, zanubrutinib, CT-1530, DTRMWXHS-12, spebrutinib besylate, vecabrutinib, evobrutinib, tirabrutinib, fenebrutinib, poseltinib, BMS-986142, ARQ-531,LOU-064, PRN-1008, ABBV-599, AC-058, ARQ-531, BMS-986195, HWH-486, PRN-2246, TAK-020, GDC-0834, BMX-IN-1, RN486, SNS-062, LFM-A13, PCI-32765 (racemate of ibrutinib), CGI-1746, ONO-4059, and SHR-1459, and their pharmaceutically acceptable salts, and preferably the BTK inhibitor is selected from M7583, ibrutinib, acalabrutinib, zanubrutinib, CT-1530, DTRMWXHS-12, spebrutinib besylate, vecabrutinib, ARQ-531, and SHR-1459, and the pharmaceutically acceptable salts thereof.

16-17. (canceled)

18. The method of claim 17, wherein enzastaurin, or a pharmaceutically acceptable salt thereof, and the inhibitor of BTK are administered together or administered separately.

19. (canceled)

20. The method of claim 19, wherein enzastaurin, or a pharmaceutically acceptable salt thereof, and the inhibitor of BTK are administered on a schedule which causes both to be present in the blood or plasma of the treated subject together.

21. The method of claim 11, wherein lymphoma is Hodgkin lymphoma or Non-Hodgkin lymphoma.

22. The method of claim 21, wherein lymphoma is non-Hodgkin lymphoma.

23. (canceled)

24. The method of claim 11, wherein enzastaurin, or a pharmaceutically acceptable salt thereof, is administered orally.

25. The method of claim 24, wherein the inhibitor of BTK is administered orally.

26. The method of claim 11, wherein the inhibitor of BTK is ibrutinib or a pharmaceutically acceptable salt thereof.

27. The method of claim 11, wherein the subject is selected based on expression or presence of a biomarker.

28. The method of claim 26, wherein the biomarker is DGM1.

29-30. (canceled)


SEQUENCE LISTING (5 sequences)

SEQ ID NO: 1 - DNA, 22 bp, Artificial Sequence
  Synthetic
tccaccagtttgaatggtcaat

SEQ ID NO: 2 - DNA, 21 bp, Artificial Sequence
  Synthetic
cgcagagggttgtattggttc

SEQ ID NO: 3 - DNA, 20 bp, Artificial Sequence
  Synthetic
gcaccgtcaaggctgagaac

SEQ ID NO: 4 - DNA, 19 bp, Artificial Sequence
  Synthetic
tggtgaagacgccagtgga

SEQ ID NO: 5 - DNA, 21 bp, Artificial Sequence
  Synthetic
tgccaacatccaggacaacat